(19) United States
(12) Patent Application Publication
(10) Pub. No.: US 2006/0205072 A1
(43) Pub. Date: Sep. 14, 2006

(54) Enriched pancreatic stem cell and progenitor cell populations, and methods for identifying, isolating and enriching for such populations

(76) Inventors: Nobuko Uchida, Palo Alto, CA (US); Ann Tsukamoto, Stanford, CA (US); Stanley Tamaki, Kensington, CA (US); Alexandra Capela, Mountain View, CA (US); Tim Austin, Morgan Hill, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/928,456

(22) Filed: Aug. 27, 2004

Related U.S. Application Data
(60) Provisional application No. 60/498,470, filed on Aug. 27, 2003.

(51) Int. Cl. C12N 5/08; A61K 39/395

(52) U.S. Cl. 435/366; 424/140.1

(57) ABSTRACT

Enriched pancreatic stem and progenitor cell populations, and methods for identifying, isolating and enriching for pancreatic stem cells using reagents that bind to cell surface markers are provided.

### RELATED APPLICATIONS

This application claims priority to U.S. Ser. No. 60/498,470, filed Aug. 27, 2003, which is herein incorporated by reference in its entirety.

### FIELD OF THE INVENTION

This invention relates generally to enriched pancreatic stem cell and progenitor cell populations, and methods for identifying, isolating and enriching for pancreatic stem and progenitor cells.

### BACKGROUND OF THE INVENTION

In the US, there are about 10.3 million diagnosed diabetes sufferers and 798,000 new cases are diagnosed annually. There are two main types of diabetes: Type I or juvenile-onset, and Type II or adult-onset diabetes. Type I diabetes, which accounts for 5-10% of all cases, is an autoimmune disease where the body attacks and destroys its own insulin-producing cells.

Treatment options for diabetes sufferers vary according to the severity of their case. Type I diabetes patients usually need to inject insulin several times daily. Diabetes patients are two to four times as likely to die from heart attack or suffer a stroke as people without diabetes. They also have an increased risk of suffering from a multitude of other diseases including kidney disease, nervous system disease, and blindness. The total annual cost of Type I diabetes in the U.S. is estimated to be $5 billion in direct medical costs and indirect costs such as disability and work loss.

Cellular therapy with stem cells and their progeny is a promising new approach capable of addressing this largely unmet medical need. The considerable excitement surrounding the stem cell field is based on the unique biological properties of these cells: their capacity to self-renew and become the component cell types of the organ in which they reside.

Stem cell populations constitute only a small percentage of the total number of cells in the body, but are of immense interest because of their ability to repopulate the body. The longevity of stem cells and the dissemination of stem cell progeny are desirable characteristics. There is significant commercial interest in these methods because stem cells have a number of clinical uses. There is also medical interest in the use of stem cells as a vehicle for gene therapy.

Proteins and other cell surface markers found on stem cell and progenitor cell populations are useful in preparing reagents for the separation and isolation of these populations. Cell surface markers are also useful in the further characterization of these important cells.

Identification of “unique” gene products expressed by pancreatic stem cells would expand the understanding of these important cells, aid in their identification in vivo and enable their positive enrichment in vitro for study and use. One useful marker would be a cell surface molecule allowing stem cell localization and purification. Thus, there remains a need for tools, such as monoclonal antibodies that are useful in isolating and characterizing human non-hematopoietic progenitor and stem cells, and particularly pancreatic stem cells and progenitor cells.

### SUMMARY OF THE INVENTION

This invention provides methods for identifying, isolating, and enriching for human non-hematopoietic progenitor and stem cells, and particularly pancreatic stem cells, progenitors, or combinations thereof, which can differentiate into somatic cells of the pancreatic lineage, including glucose-responsive, insulin secreting cells. The invention also provides for enriched populations containing pancreatic stem cells and progenitor cells.

Enriched populations of non-hematopoietic stem cells and progenitor cells, preferably pancreatic stem cells and/or progenitors, and methods of identifying, isolating, or enriching for such cells, are achieved by contacting a population of cells containing at least one stem cell or progenitor cell with a reagent that binds to a surface marker glycoprotein antigen (“CD49f antigen”) recognized by an antibody that specifically binds to CD49f (“anti-CD49f antibody”) or to a cell surface marker antigen (“CD133 antigen”) recognized by an antibody that specifically binds to CD133 (“anti-CD133 antibody”). As used herein, the term “reagent” is meant to include any composition or compound that is capable of binding to, associating with, or recognizing an antigen. Examples of such reagents include, but are not limited to monoclonal antibodies, polyclonal antibodies, small molecules, receptors, ligands, proteins, protein fragments, polypeptides, polypeptide fragments, nucleic acids, nucleic acid fragments, antibody fragments, and any other “reagents” known to those skilled in the art.

While the methods described herein refer to the use of the CD49f antigen and/or the CD133 antigen to enrich populations of pancreatic cells or gastrointestinal cells for pancreatic stem cells or progenitor cells or gastrointestinal stem cells or progenitor cells, those skilled in the art will recognize that any other cell-surface marker present on pancreatic stem cells or progenitors can also be used in the methods of the instant invention. Additionally, those skilled in the art will recognize that any suitable cell surface markers can be use in any order and/or in any combination. CD133⁺ cells are defined as cells containing the CD133 antigen and CD49f⁺ cells are defined as cells containing the CD49f antigen. Moreover, the skilled artisan will recognize that any combination of CD49f and CD133 and/or antigens, in any order, can be used to produce populations of pancreatic cells enriched for pancreatic stem cells. Those skilled in the art will also recognize that any reference to anti-CD133 and/or anti-CD49f antibodies encompasses human, murine, rat, sheep, equine, goat, chicken, rabbit, guinea pig, and/or porcine antibodies.

Use of traditional techniques for cell sorting, such as by immunoselection (e.g., FACS), permits identification, isolation, and/or enrichment for cells in which contact between the reagent and the CD49f antigen and/or the CD133 antigen has been detected. The reagent can be an anti-CD49f antibody (two such anti-CD49f antibodies are referred to herein as “GoH3” and “4F10”) or an anti-CD133 antibody (two such anti-CD133 antibodies are referred to herein as “AC133” and “SC111”).

This invention also provides methods of using antibodies to provide enriched populations of non-hematopoietic stem cells and progenitor cells, preferably pancreatic stem cells, progenitor cells, or combinations thereof that may be used in methods of identifying, isolating, or enriching for such cells, by contacting a population of cells containing at least one stem cell or progenitor cell with an anti-CD49f antibody or with an anti-CD133 antibody. The methods of the invention can be used to enrich for human pancreatic stem cells, progenitors, or a combination thereof, by contacting pancreatic tissue, pancreatic cells, pancreatic-derived cells, or primary gastrointestinal tissue or gastrointestinal-derived cells with an appropriate reagent, e.g. an antibody.

The cells of this invention, preferably the pancreatic stem cells or progenitor cells, are additionally characterized as lacking cell surface markers for CD45 and CD34 (e.g., the CD45⁻ and CD34⁻ phenotypes). As such, purified populations can be achieved by removing cells that are CD45⁻ and CD34⁻ from pancreatic tissue, pancreatic cells, pancreatic-derived cells, or primary gastrointestinal tissue or gastrointestinal-derived cells or by selecting for cells that are CD45⁺ and CD34⁺.

Methods of producing a population from pancreatic tissue enriched for human pancreatic stem cells, progenitors, or a combination thereof are provided. Such methods involve the steps of contacting pancreatic tissue, pancreatic cells, pancreatic-derived cells, or primary gastrointestinal tissue or gastrointestinal-derived cells with a monoclonal antibody that binds CD133 or CD49f, and selecting said pancreatic tissue, pancreatic cells, pancreatic-derived cells, or primary gastrointestinal tissue or gastrointestinal-derived cells that bind to the monoclonal antibody, wherein the selected cells are enriched for human pancreatic stem cells, progenitors, or a combination thereof. The population containing pancreatic stem/progenitor cells, is obtained from a suspension culture or an adherent culture, from any tissue which gives rise to pancreatic tissue, and/or from primary gastrointestinal tissue or gastrointestinal derived explant.

Additionally, such methods may also involve the steps of further enriching a population from pancreatic or gastrointestinal tissue for pancreatic stem cells, progenitors, or combinations thereof by contacting the selected cells with a second monoclonal antibody that binds CD34 or CD45; and removing those cells that are CD34⁺ or CD45⁺, wherein the remaining cells in the population are CD34⁻ or CD45⁻ and are enriched for pancreatic stem cells, progenitors, or combinations thereof. Moreover, those skilled in the art will recognize that the pancreatic tissue, pancreatic cells, pancreatic-derived cells, or primary gastrointestinal tissue or gastrointestinal-derived cells are contacted with an anti-CD34 monoclonal antibody or an anti-CD45 monoclonal antibody, and wherein the bound cells are removed prior to the contacting with a monoclonal antibody that binds CD133 or CD49f.

The invention also provides methods for producing a population enriched for human pancreatic stem cells, progenitors, or a combination thereof, comprising selecting from a population of pancreatic tissue, pancreatic cells, pancreatic-derived cells, or primary gastrointestinal tissue or gastrointestinal-derived cells for those cells that are CD133⁺, CD49f⁺, or CD133⁺ CD49f⁺. Such selection may be accomplished by contacting the population of cells with an anti-CD133 antibody selected from the group consisting of monoclonal antibody AC133 and monoclonal antibody SC111 and removing those cells that do not bind to the anti-CD133 antibody or by contacting the population of cells with an anti-CD49f antibody selected from the group consisting of monoclonal antibody GoH3 and monoclonal antibody 4F10 and removing those cells that do not bind to the anti-CD49f antibody. The remaining populations can be further enriched by removing the cells that are CD34⁺ (i.e. those cells that bind to a monoclonal antibody that recognizes CD34) and/or by removing the cells that are CD45⁺ (i.e. those cells that bind to a monoclonal antibody that recognizes CD34) from the remaining population.

The invention also provides methods for enriching from a population of pancreatic tissue, pancreatic cells, pancreatic-derived cells, or primary gastrointestinal tissue or gastrointestinal-derived cells for the populations of pancreatic stem cell or progenitor fractions, comprising selecting from the pancreatic tissue, pancreatic cells, pancreatic-derived cells, or primary gastrointestinal tissue or gastrointestinal-derived cells for cells that express CD133 by binding to monoclonal antibody AC133 or monoclonal antibody SC111, wherein the selected cells are enriched in the fraction of pancreatic stem cells as compared with the population of pancreatic tissue, pancreatic cells, pancreatic-derived cells, or primary gastrointestinal tissue or gastrointestinal-derived cells. Again, this fraction can be further enriched by removing cells that are CD34⁺ and/or by removing cells that are CD45⁺.

Moreover, methods are provided for enriching for pancreatic stem cell or progenitor cell fractions, comprising selecting from the pancreatic tissue, pancreatic cells, pancreatic-derived cells, or primary gastrointestinal tissue or gastrointestinal-derived cells for cells that express CD49f by binding to monoclonal antibody GoH3 or monoclonal antibody 4F10, wherein the selected cells are enriched in the fraction of pancreatic stem cells as compared with the population of pancreatic tissue, pancreatic cells, pancreatic-derived cells, or primary gastrointestinal tissue or gastrointestinal-derived cells. Again, this fraction can be further enriched by removing cells that are CD34⁺ and/or the cells that are CD45⁺.

Methods for isolating a pancreatic stem cell from primary pancreatic tissues may include the steps of selecting from a population pancreatic cells, pancreatic-derived cells, or gastrointestinal-derived cells for cells that are CD133⁺, CD49f⁺, or CD133⁺CD49f⁺ and removing the cells that are CD34⁺, CD45⁺, or CD34⁺CD45⁺, wherein the remaining cells are CD34⁺, CD45⁻ or CD34⁻CD45⁻; introducing the cells remaining to a serum-free culture medium containing one or more growth factors; and proliferating the remaining cells in the culture medium.

The invention also provides methods for producing a population enriched for human pancreatic stem cells, progenitors, or combinations thereof comprising selecting from pancreatic tissue, pancreatic cells, pancreatic-derived cells, or primary gastrointestinal tissue or gastrointestinal-derived cells for cells that are CD133⁺ and bind to an anti-CD133 antibody selected from the group consisting of monoclonal antibody AC133 and monoclonal antibody SC111, to produce a population enriched for pancreatic stem cells, progenitors, or combinations thereof, wherein the selecting is by attachment to and disattachment from solid phase. Similarly, such methods may include selection for cells that are CD49f⁺ and bind to an anti-CD49f antibody selected from the group consisting of monoclonal antibody GoH3 and monoclonal antibody 4F10.

The invention also provides antibodies that specifically bind to the CD49f antigen, wherein said CD49f antigen specifically binds to the monoclonal antibody GoH3 or to the monoclonal antibody 4F10. This monoclonal antibody may be produced by a hybridoma cell line and may bind to the CD49f antigen as detected by the GoH3 antibody or the 4F10 antibody. Likewise, antibodies that specifically bind to the CD133 antigen, wherein said CD133 antigen specifically binds to the monoclonal antibody AC133 or to the monoclonal antibody SC111 are also provided. This monoclonal antibody may be produced by a hybridoma cell line and may bind to the CD133 antigen as detected by the AC133 antibody or the SC111 antibody.

Moreover, methods are provided for enriching for human pancreatic stem cells, progenitors, or a combination thereof by combining a population of pancreatic tissue, pancreatic cells, pancreatic-derived cells, or primary gastrointestinal tissue or gastrointestinal-derived cells with a reagent that specifically binds to the CD133 antigen, the CD49f antigen or both the CD133 and CD49f antigens; and selecting for those cells that bind to the reagent, wherein the selected cells are enriched for pancreatic stem cells, progenitors, or a combination thereof, as compared to the population. For example, the reagent may be at least one antibody.

Methods for producing population enriched for human pancreatic stem cells, progenitors, or a combination thereof, by selecting from pancreatic tissue, pancreatic cells, pancreatic-derived cells, or primary gastrointestinal tissue or gastrointestinal-derived cells for those cells that express CD133 and bind to monoclonal antibody AC133 or to monoclonal antibody SC111, (or cells that express CD49f and bind to monoclonal antibodies GoH3 or 4F10) to produce a population enriched for pancreatic stem cells, progenitors, or a combination thereof.

In any of the methods described herein, the population containing pancreatic tissue, pancreatic cells, pancreatic-derived cells, or primary gastrointestinal tissue or gastrointestinal-derived cells is obtained from a suspension culture, an adherent monolayer culture, a pancreatic explant, a gastrointestinal explant, from primary pancreatic tissue, or from primary gastrointestinal tissue.

Additionally, methods are included for producing a population enriched for human pancreatic stem cells, progenitors, or a combination thereof, wherein the population is obtained from primary pancreas tissues, by selecting from a population of pancreatic tissue, pancreatic cells, pancreatic-derived cells, or primary gastrointestinal tissue or gastrointestinal-derived cells for cells that are CD133⁺, CD49f⁺, or CD133⁺CD49f⁺ the method further comprising the steps of further enriching for pancreatic stem cells, progenitors, or a combination thereof, by further selecting for those cells that are CD34⁻, CD45⁻, or CD34⁻CD45⁻.

Isolation of pancreatic stem cells may be achieved by selecting from a population of primary pancreas tissues, from a population of pancreatic tissue, pancreatic cells, pancreatic-derived cells, or primary gastrointestinal tissue or gastrointestinal-derived cells for at least one selected cell that binds to a monoclonal antibody selected from the group consisting of: AC133, SC111, GoH3, and 4F10; introducing at least one selected cell to a serum free culture medium containing one or more growth factors; and proliferating the at least one selected cell in the culture medium. The tissue or cells may be obtained from a suspension culture or an adherent culture. Additionally, the tissue or cells may be obtained from human fetal, neonatal, juvenile, adult, or postmortem pancreas tissue. Moreover, the selected cells can be further enriched by contacting the selected cells with a second monoclonal antibody that binds to CD34 antigen or CD45 antigen; and removing those cells that are CD34⁺, CD45⁺, or CD34⁺CD45⁺ from the population, wherein the remaining cells in the population are enriched for pancreatic stem cells, progenitors, or a combination thereof. For example, the tissue or cells can be contacted with a monoclonal antibody that binds to CD34 antigen or a monoclonal antibody that binds to CD45 antigen.

The invention also provides methods of producing a population from pancreatic tissue enriched for human pancreatic lineage committed progenitor and mature cells, by contacting pancreatic cells, pancreatic-derived cells, or gastrointestinal-derived cells with a monoclonal antibody that binds CD9, and selecting said primary pancreas tissues, the method comprising selecting from a population of pancreatic tissue, pancreatic cells, pancreatic-derived cells, or primary gastrointestinal tissue or gastrointestinal-derived cells that bind to the monoclonal antibody, wherein the selected CD9⁺ cells are enriched for human pancreatic β-cell lineage committed progenitor or mature cells capable of differentiating into insulin⁺ β cells. The remaining cells can also be further enriched by contacting the selected cells with a second monoclonal antibody that binds CD15 and removing those cells that are CD15⁺, wherein the remaining cells in the population are CD15⁻ and are enriched for human pancreatic β-cell lineage committed progenitor cells capable of differentiating into insulin⁺ β cells. Such selection can be accomplished using an anti-CD15 monoclonal antibody and wherein the bound cells are removed.

The invention also provides methods for producing a population enriched for human pancreatic lineage committed progenitor cells, comprising selecting from a population of primary pancreas tissues by selecting from a population of pancreatic tissue, pancreatic cells, pancreatic-derived cells, or primary gastrointestinal tissue or gastrointestinal-derived cells for those cells that are CD49f⁺⁺CD9⁺ or CD133⁺CD9⁺.

Isolation of pancreatic stem cells from primary pancreatic tissue occurs by selecting from a population of pancreatic cells, pancreatic-derived cells, or gastrointestinal-derived cells for cells that are CD133⁺, CD49f⁺, or CD133⁺CD49f⁺; removing the cells that are CD15⁺, wherein the remaining cells are CD15⁻; introducing the remaining cells to a serum-free culture medium containing one or more growth factors; and proliferating the remaining cells in the culture medium.

Also provided are methods for producing populations enriched for human pancreatic stem cells, wherein the population is obtained from primary tissues, by selecting from a population of pancreatic tissue, pancreatic cells, pancreatic-derived cells, or primary gastrointestinal tissue or gastrointestinal-derived cells that are CD133⁺, CD49f⁺, or CD133⁺CD49f⁺ and by further enriching for pancreatic stem cells, by further selecting for those cells that are CD15⁻

Production of a population enriched for human pancreatic stem cells, progenitors, or a combination there of can also be achieved by further enriching the population by removing those cells that are CD49f⁻, wherein the remaining cells in the population are CD49f⁺ and are enriched for pancreatic stem cells, progenitors, or combinations thereof.

The invention also provides a pancreatic stem cell that is CD133⁺CD49f⁺. Also provided is a pancreatic progenitor cell committed to the endocrine β-cell lineage, wherein the progenitor cell is CD49f⁺CD9⁺. This cell may also be CD15⁻. Also provided is a pancreatic progenitor cell committed to the endocrine β-cell lineage, wherein the progenitor cell is CD49f⁺. The invention also provide a mature β-cell lineage pancreatic cell, wherein the pancreatic cell is CD133⁻CD49f⁻ CD9⁻CD15⁻. This cell is insulin⁺.

Pancreatic stem cells can produce progeny that include several different types of progenitor cells. Some pancreatic progenitor cells of the endocrine lineage are express the CD9 antigen, while others express the CD15 antigen. Endocrine β lineage committed progenitor cells include progenitors that lack the CD133 antigen and the CD15 antigen and that express the CD49f antigen and the CD9 antigen (i.e., the CD133⁻CD49f⁺CD15⁻CD9⁺ phenotype). Pancreatic cells that are CD133⁻CD49f⁻CD9⁻CD15⁻ are mature insulin-producing β cells.

The invention also involves methods for producing a population enriched for human pancreatic stem cells and/or progenitors by contacting pancreatic cells or pancreatic-derived cells with a monoclonal antibody that binds to CD49f or with a monoclonal antibody that binds to CD133; and selecting the cells that bind to this monoclonal antibody; wherein the selected cells are enriched for human pancreatic stem cells and/or progenitors. In various embodiments, the monoclonal antibody may be fluorochrome conjugated or may be conjugated to magnetic particles. Additionally, the selecting may be by fluorescence activated cell sorting, high gradient magnetic selection, or by attachment to and disattachment from the solid phase. The population containing pancreatic cells or pancreatic-derived cells can be obtained from a suspension culture, or an adherent culture.

Alternatively, the selection methods of the invention may also involve the step of enriching the population obtained from primary pancreas tissue for pancreatic stem cells and/or progenitors by contacting the removed cells with a monoclonal antibody that binds to CD45 or CD34 and eliminating those cells that bind to the monoclonal antibody to produce a population enriched for pancreatic stem cells and/or progenitors. Such methods may also involve the step of further enriching the population for pancreatic stem cells and/or progenitors by contacting the remaining cells with an anti-CD133 or an anti-CD49f monoclonal antibody and selecting those cells that bind to the monoclonal antibody to obtain a population enriched for pancreatic stem cells and/or progenitors.

Also provided is a monoclonal antibody (SC111), which recognizes the CD133 antigen, wherein said monoclonal antibody was deposited at the American Type Tissue Collection on ______, and give the ATCC designation ______.

Unless otherwise defined, all technical and scientific terms used herein have the same meaning as commonly understood by one of ordinary skill in the art to which this invention belongs. Although methods and materials similar or equivalent to those described herein can be used in the practice of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the invention will be apparent from the following detailed description and claims.

### BRIEF DESCRIPTION OF THE DRAWINGS

FIG. 1 is a photograph of the immunohistochemical staining of fetal pancreas tissue for the CD133 and CD49f cell surface markers. CD133 is expressed on the luminal surface of ductal cells. CD49f is expressed on the apical surface of ductal cells.

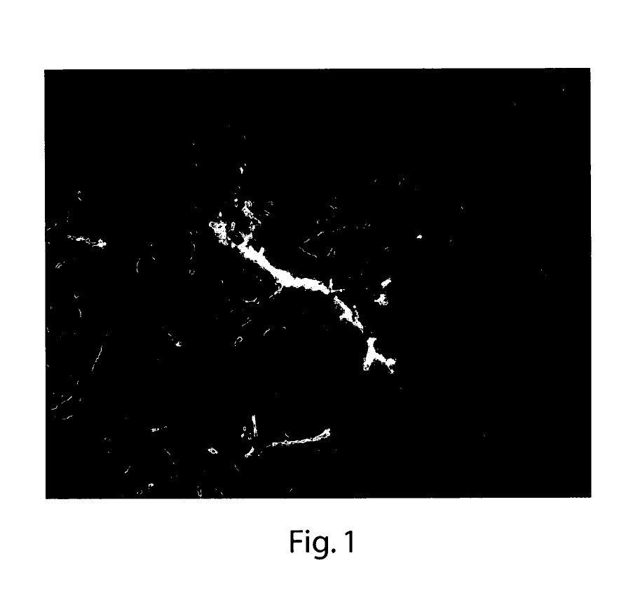

FIG. 2 is a photograph of the immunohistochemical staining of adult pancreas tissue. CD133 expression is observed on the luminal surface of ductal cells.

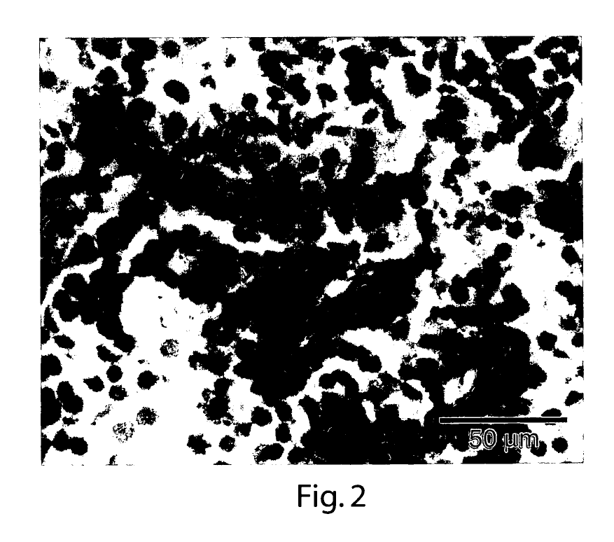

FIG. 3 is a photograph of the immunohistochemical staining of fetal pancreas tissue showing that, in most embodiments, CD133 expression and insulin expression is mutually exclusive.

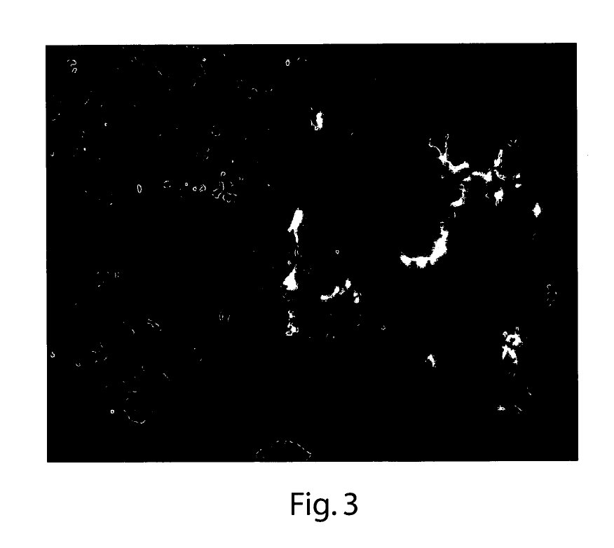

FIG. 4 is a photograph of the immunohistochemical staining of fetal pancreas tissue showing that, in some embodiments, the same cells co-express insulin and CD49, as confirmed by microscopic analysis (arrows).

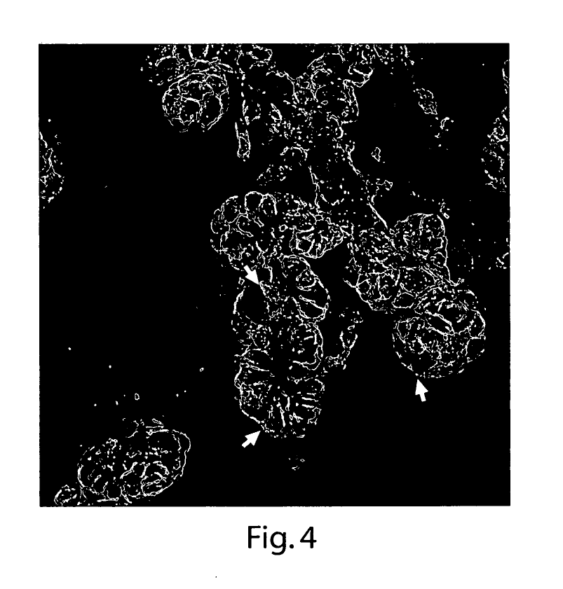

FIG. 5 is a photograph of the immunohistochemical staining of fetal pancreas tissue showing that, in some embodiments, the same cells co-express insulin and CK19, as confirmed by microscopic analysis (arrows).

FIG. 6 is a photograph of the immunohistochemical staining of fetal pancreas tissue showing that, in some embodiments, the same cells co-express insulin and CD9, as confirmed by microscopic analysis (arrows).

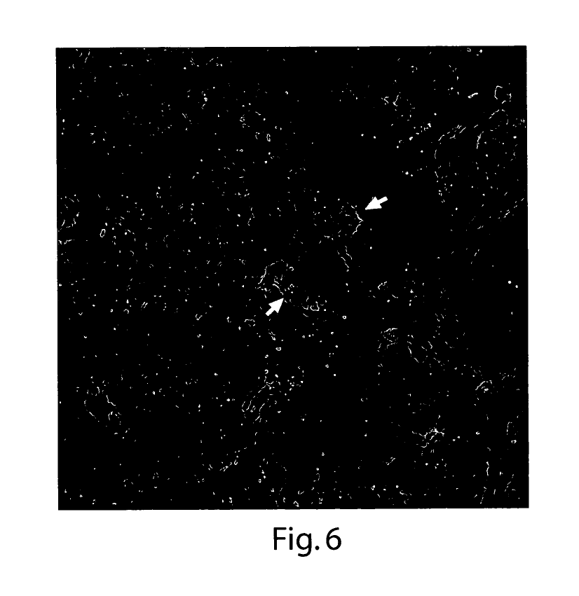

FIG. 7 is a photograph of the co-immunohistochemical staining of fetal pancreas tissue for the CD15 cell surface marker and insulin. CD15⁺ cells and insulin⁺ cells are mutually exclusive.

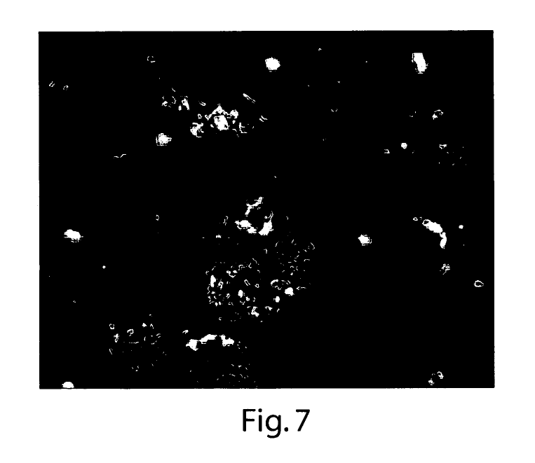

FIG. 8 is a photograph of the co-immunohistochemical staining of fetal pancreas tissue for the CD9 cell surface marker and insulin.

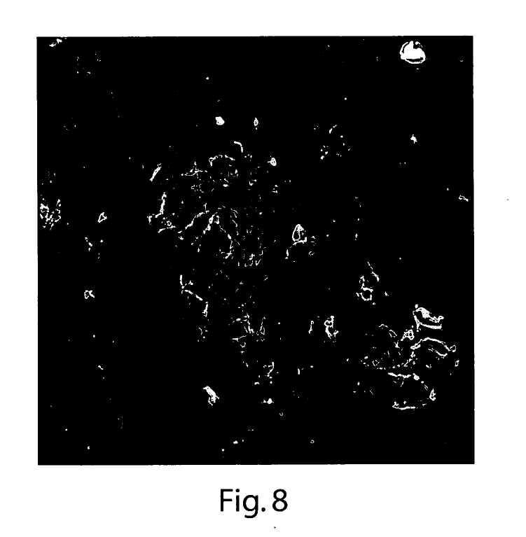

FIGS. 9A and 9B are graphs showing FACS analysis pancreatic cells based on cell surface markers. CD133⁺CD49f⁺ cells are enriched for pancreatic stem/progenitor cells (FIG. 9A). Differentiating α-cells and β-cells are separated into CD15⁺CD9⁻ and CD9⁺ CD15⁻ cell populations, respectively (FIG. 9B). These pancreatic cells are stained with antibodies against CD34 and CD45, and CD45+ hematopoeitic cells and CD34+ endothelial cells are excluded from these analyses.

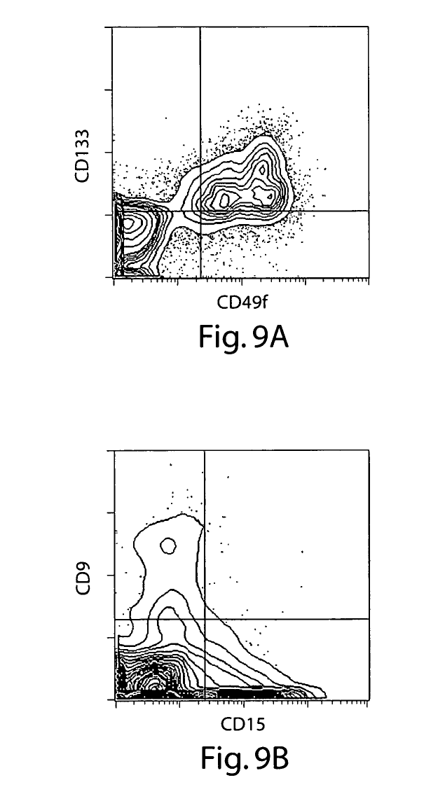

FIG. 10 is a diagram depicting the antigen markers for pancreatic stem cells and their progeny, including mature ductal cells, progenitor cells, and terminally differentiated cells of the endocrine lineage.

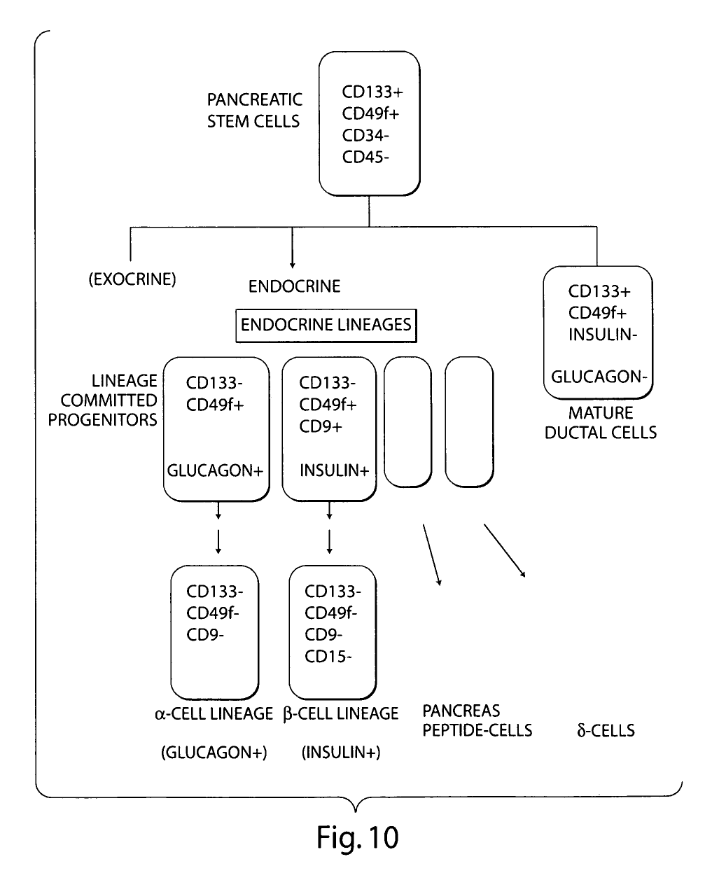

### DETAILED DESCRIPTION OF THE INVENTION

Pancreatic exocrine and endocrine cells develop from precursor cells present in the pancreas or the gastrointestinal tract. Embryonic endocrine cells aggregate and form the islets of Langerhans, which, in mice, achieve a typical adult configuration after birth. Insulin-containing β cells form the core of the mature islets, whereas the periphery contains lower numbers of the other endocrine cell types: the α, δ, and PP cells, which synthesize glucagons, somatostatin, and pancreatic polypeptide respectively.

A population of cells exists within the adult pancreas which exhibit stem cell properties. They have the ability to self-renew and to produce the differentiated mature cell phenotypes of adult pancreas tissue or gastrointestinal tissue.

Undifferentiated, multipotent, self-renewing, pancreatic cells are termed “pancreatic stem cells.” A pancreatic stem cell is a clonogenic multipotent stem cell, which is able to divide and, under appropriate conditions, has self-renewal capability and can include in its progeny daughter cells, which can terminally differentiate into mature cells of the pancreas. Hence, the pancreatic stem cell is “multipotent” because stem cell progeny have multiple differentiation pathways. A pancreatic stem cell is capable of self maintenance, meaning that with each cell division, one daughter cell will also on average be a stem cell.

The non-stem cell progeny of a pancreatic stem cell are typically referred to as “progenitor” cells, which are capable of giving rise to various cell types within one or more lineages. The term “pancreatic progenitor cell” refers to an undifferentiated cell derived from a pancreatic stem cell, and is not itself a stem cell. Some progenitor cells can produce progeny that are capable of differentiating into more than one cell type. A distinguishing feature of a progenitor cell is that, unlike a stem cell, it does not exhibit self maintenance, and typically is thought to be committed to a particular path of differentiation.

Pancreatic progenitors may be characterized by the expression of one or more of: homeodomain type transcription factors, such as STF-1; and PAX genes, such as PAX6, PTF-1, hXBP-1, HNF genes, villin, tyrosine hydroxylase, insulin, glucagons, and/or neuropeptide Y. The pancreatic progenitor cells of the present invention may also be characterized by binding to lectins, such as plant lectin or peanut agglutinin. The progenitor cells may also express PDX1 mRNA and protein, and be capable of differentiation into glucose-responsive insulin secreting cells. Alternatively, the progenitor cells may express Glut2 mRNA and protein.

In general, the progenitor cells of the present invention are proliferative cells which can differentiate into cells making up the tissues of the pancreas or the gastrointestinal tract. That is, the progenitor cells can give rise to differentiated cells of pancreatic lineages. For example, the progenitor cells are inducible to differentiate into pancreatic islet cells, e.g., β islet cells, a islet cells, δ islet cells, or φ islet cells (or PP cells).

As used herein, the term “precursor cells” refers to the progeny of pancreatic stem cells, and thus includes both progenitor cells and daughter stem cells.

Cell markers. This invention provides for the identification, isolation, enrichment, and culture of pancreatic stem cells and/or progenitors. Pancreatic stem cells and/or progenitors are identified or selected through the binding of antigens, found on the surfaces of pancreatic stem cells and/or progenitors, to reagents that specifically bind the cell surface antigen. Also provided are methods for identification, isolation, enrichment, and culture of glucagon-producing (glucagon⁺) cells and insulin-producing (insulin⁺) cells.

In order to normalize the distribution to a control, each cell is recorded as a data point having a particular intensity of staining. These data points may be displayed according to a log scale, where the unit of measure is arbitrary staining intensity. In one example, the brightest cells in a population are designated as 4 logs more intense than the cells having the lowest level of staining. When displayed in this manner, it is clear that the cells falling in the highest log of staining intensity are bright, while those in the lowest intensity are negative. The “low” staining cells, which fall in the 2-3 log of staining intensity, may have properties that are unique from the negative and positive cells. An alternative control may utilize a substrate having a defined density of marker on its surface, for example a fabricated bead or cell line, which provides the positive control for intensity. The “low” designation indicates that the level of staining is above the brightness of an isotype matched control, but is not as intense as the most brightly staining cells normally found in the population.

As used herein, the terms CD133^{lo}, CD133^{low}, and/or CD133^{-/lo} refer to “low” staining cells, which fall into the 1^{st}-2^{nd} log of staining intensity. When a few molecules (<100-500) in a given antigen were expressed on the cell surface, the signal to noise ratio may be too poor to determine whether a given antigen is expressed on the cell surface. Those skilled in the relevant arts will recognize that any of the antibodies described herein can also be described using the “lo” or “low” designation (i.e. antibodyX^{low} or antibodyX^{low}), without altering the intended meaning. Likewise, as used herein, the terms CD133^{hi}, CD133^{high}, and/or CD133^{bright} refer to those cells in the population designated as 3 logs more intense than the cells having the lowest level of staining. Again, those skilled in the art will recognize that any antibody can be described using these designations, without altering the intended meaning (i.e., antibodyX^{hi}, antibodyX^{high}, or antibodyX^{bright}). The designation antibodyX^{med} is intended to refer to an antibody having a staining intensity falling between “low” and “bright”. Moreover, as used herein, the designations antibodyX⁺ and antibodyX^{hi} are used interchangeably.

One of the antigens found on the surface of pancreatic stem cells and/or progenitors is an antigen that binds to the AC133 monoclonal antibody (i.e., the CD133 antigen). Yin et al., U.S. Pat. No. 5,843,633, incorporated herein by reference, describes a monoclonal antibody called AC133, which binds to a surface marker glycoprotein on hematopoietic stem and progenitor cells. The AC133 antigen (also referred to herein as the “CD133 antigen” or “CD133”) is a 5-transmembrane cell surface antigen with a molecular weight of 117 kDa. Expression of this antigen is highly tissue specific, and has been detected on a subset of hematopoietic progenitor cells derived from human bone marrow, fetal bone marrow and liver, cord blood, and adult peripheral blood. The subset of cells recognized by the AC133 antibody is CD34^{bright}, and contains substantially all of the CFU-GM activity present in the CD34⁺ population, making AC133 useful as a reagent for isolating and characterizing human hematopoietic progenitor and stem cells.

The AC133 antibody (also referred to herein as the 5F3 antibody) is exemplary of antibody reagents that recognize a human cell marker termed prominin. Prominin is a polytopic membrane protein expressed in various epithelial cells (Weigmann et al., 94(23) Proc Natl Acad Sci USA. 12425-30 (1997); Corbeil et al., 112 (Pt 7) J Cell Sci. 1023-33 (1999); Corbeil et al., 91(7) Blood 2625-6 (1998); Miriglia et al., 91(11) Blood 4390-1 (1998)). Various AC133 antibodies are described in U.S. Pat. No. 5,843,633, which is incorporated herein by reference. A deposit of the murine hybridoma cell line AC133 was made at the American Type Tissue Collection, 12301 Parklawn Drive, Rockville Md. 20852, on Apr. 24, 1997, and given the ATCC designation HB12346. These AC133 antibodies are capable of immunoselection for a subset of human cells of interest in this invention. Preferred AC133 monoclonal antibodies can be obtained commercially from Miltenyi Biotec Inc. (Auburn Calif.), including, but not limited to, AC133/1-PE antibody (Cat #808-01) and AC133/2-PE antibody (Cat #809-01). For MACS separation, a 50:50 mixture of the monoclonal antibodies is preferred. The high tissue specificity of AC133 expression is particularly advantageous during enrichment for highly purified pancreatic stem cells and/or progenitors populations. A discussion of the use of the AC133 antigen to select NS-IC is found in U.S. Pat. No. 6,468,794, which is incorporated herein by reference.

SC111 (StemCells, Inc., Palo Alto, Calif.) deposited at the American Type Tissue Collection on ______, and give the ATCC designation ______, is another antibody that binds to the CD133 antigen. Pancreatic stem cells may be classified as CD133⁺.

CD45 is the T200/leucocyte common antigen. Antibodies to CD45 are commercially available from, e.g. Miltenyi Biotec (Auburn, Calif.) (catalog numbers 130-080-201; 130-080-202); and Research Diagnostics (Flanders, N.J.) (catalog numbers RDI-M1343clb; RDI-CBL124; RDI-CBL148; RDI-CBL464, etc.). In a preferred embodiment, the cells of this invention and cultures containing them, are additionally characterized (in addition to being prominin positive) as lacking cell surface markers such as CD45. Pancreatic stem cells or progenitors are typically CD45⁻.

CD34 is also known as gp105-120. Monoclonal antibodies to CD34 are commercially available from, e.g., Miltenyi Biotec (Auburn, Calif.) (catalog numbers 130-090-954); Research Diagnostics (Flanders, N.J.) (catalog numbers RDI-M1636clb; RDI-CBL128; RDI-CBLA96FT; RDI-M2281clb; RDI-CD34-581, etc.); BD Biosciences, Pharmingen (San Diego, Calif.) (catalog number 550760)). Anti-CD34 monoclonal antibodies have been used to quantify and purify lymphohematopoietic stem/progenitor cells for research and for clinical bone marrow transplantation. CD34 is a monomeric cell surface antigen with a molecular mass of approximately 110 kDa that is selectively expressed on human progenitor cells. The gene is expressed by small vessel endothelial cells in addition to hematopoietic progenitor cells and is a single-chain 105-120 kDa heavily O-gylcosylated transmembrane glycoprotein. The sequence is disclosed by Simons et al. (1992) J. Immun. 148:267-271. Pancreatic stem cells or progenitors are typically CD34⁻.

CD49f (also known as integrin alpha-6) (GenBank Accession No. X53586; SWISSPROT Accession No. P23229) is a 150 kDa transmembrane protein that is part of an integrin heterodimer expressed predominantly by epithelial cells. Integrin alpha-6 associates with the integrin β-1 (CD29) chain to form VLAA-6 and with the integrin β-4 chain to form the laminin and kalinin receptors. CD49f is expressed mainly on T cells, monocytes, platelets, epithelial and endothelial cells, perineural cells, and trophoblasts of placenta. The sequence of CD49f may be found in, e.g., Tamura et al., J. Cell Biol. 111:1593-604 (1990), which is incorporated herein by reference. There are two alternatively spliced forms of CD49f cDNA, which have been described as having different cytoplasmic domains. The A form alone is expressed in the lung, liver, spleen, and cervix. Only the B form is observed in the brain, ovary, and kidney, and both forms have been detected in other tissues. CD49f/CD29 α6β1 is the laminin receptor on platelets, monocytes, and T lymphocytes, and CD49f/CD29-mediated T cell binding to laminin provides a co-stimulatory signal to T cells for activation and proliferation.

Antibodies to CD49f have not been used in methods for identifying, isolating, or enriching for non-hematopoietic stem cells or progenitor cells, particularly pancreatic stem cells and progenitor cells. Pancreatic stem cells or progenitors may be classified as CD49f⁺.

The sequence of CD49f is presented below in Table A. Alpha-6 associates with the integrin β-1 (CD29) chain to form VLAA-6 and with the integrin β-4 claim to form the laminin and kalinin receptors. Antibodies that recognize CD49f include GoH3 [Research Diagnostics, Inc., Flanders, N.J. (catalog numbers RDI-M1566 and RDI-M1672clb); BD Biosciences (www.bdbiosciences.com) (catalog numbers 55710, 557511, 551140, 551129, 555734, 555735, 555736); and ICN Biomed (www.incbiomed.com)] and 4F10 [Research Diagnostics, Inc., Flanders, N.J. (catalog number RDI-CBLA58)].

TABLE A
SEQUENCE OF CD49f (SEQ ID NO:1)

| | |
|---|---|
| 1 | maaagqlcll ylsagllsrl gaafnldtre dnvirkygdp gslfgfslam hwqlqpedkr |
| 61 | lllvgaprge alplqranrt gglyscdita rgpctriefd ndadptsesk edqwmgvtvq |
| 121 | sqgpggkvvt cahryekrqh vntkqesrdi fgrcyvlsqn lrieddmdgg dwsfcdgrlr |
| 181 | ghekfgscqq gvaatftkdf hyivfgapgt ynwkgivrve qknntffdmn ifedgpyevg |
| 241 | getehdeslv pvpansylgl lfltsvsytd pdqfvyktrp preqpdtfpd vmmnsylgfs |
| 301 | ldsgkgivsk deitfvsgap ranhsgavvl lkrdmksahl lpehifdgeg lassfgydva |
| 361 | vvdlnkdgwq divigapqyf drdgevggav yvymnqqgrw nnvkpirlng tkdsmfgiav |
| 421 | knigdinqdg ypdiavgapy ddlgkvfiyh gsangintkp tqvlkgispy fgysiagnmd |
| 481 | ldrnsypdva vgslsdsvti frsrpviniq ktitvtpnri dlrqktacga psgiclqvks |
| 541 | cfeytanpag ynpsisivgt leaekerrks glssrvqfrn qgsepkytqe ltlkrqkqkv |
| 601 | cmeetlwlqd nirdklrpip itasveiqep ssrrrvnslp evlpilnsde pktahidvhf |
| 661 | lkegcgddnv cnsnlkleyk fctregnqdk fsylpiqkgv pelvlkdqkd ialeitvtns |
| 721 | psnprnptkd gddaheakli atfpdtltys ayrelrafpe kqlscvanqn gsqadcelgn |
| 781 | pfkrnsnvtf ylvlsttevt fdtpdldinl klettsnqdn lapitakakv vielllsvsg |
| 841 | vakpsqvyfg gtvvgeqamk sedevgslie yefrvinlgk pltnlgtatl niqwpkeisn |
| 901 | gkwllylvkv eskglekvtc epqkeinsln lteshnsrkk reitekqidd nrkfslfaer |
| 961 | kyqtlncsvn vncvnircpl rgldskasli lrsrlwnstf leeysklnyl dilmrafidv |
| 1021 | taaaenirlp nagtqvrvtv fpsktvaqys gvpwwiilva ilagilmial lvfilwkcgf |
| 1081 | fkrsryddsv pryhavrirk eereikdeky idnlekkqwi tkwnrnesys |

CD15 (also known as Lewis X, or LeX) (GenBank Accession No. NM 002033) is a 220 kDa branched pentasaccharide. The CD15 carbohydrate epitope is expressed in mature human neutrophils, monocytes, and eosinophils, as well as in adult mouse subventricular zone (SVZ) stem cells. It can also be found present on embryonic tissues and adenocarcinomas, myeloid leukemias and Reed-Sternberg cells. In such tissues, the Lewis X epitope is considered to be involved in cell-cell interactions. CD15 is carried by the CD11/CD18 and CD66 glycoproteins. CD15 antibodies recognize the terminal trisaccharide structure Galβ1→4[Fucα1→3]GlcNAc (LeX antigen). The majority of the CD15 antibodies are IgM, and they do not cross react with the sialylated form of CD15, CD15s. CD15 is a fucose-containing trisaccharide widely distributed in many tissues and is developmentally expressed in some rodent and human tissues, i.e., brain and lung, and mouse early embryo. Additionally, CD15 is present on the surface of pluripotent stem cells, such as mouse embryonic stem cells and primordial germ cells. The sequence of CD15 is presented in Table B. CD15 is useful as a cell type marker since it allows for stem cell localization and purification. Antibodies that recognize human CD15 include MMA (BD Biosciences (www.bdbiosciences.com) (catalog numbers 340703, 340850, 347420, 347423, 559045)).

Cell surface carbohydrate moieties are useful cell type markers (Jessell et al., (1990) Ann. Rev. Neurosci 13, 227-55). The LeX antigen, which is the trisaccharide 3-fucosyl-N-acetyllactosamine or FAL (Gooi et al., (1981) Nature 292, 156-58), also known as SSEA-1 (stage specific embryonic antigen 1) or CD15 (leukocyte cluster of differentiation 15), is highly expressed on pluripotent stem cells: it is found on mouse and human embryonic carcinoma cells, mouse pre-implantation embryos, embryonic stem cells, teratocarcinoma cells and primordial germ cells (Solter and Knowles, (1978) Proc. Natl. Acad. Sci. USA 75, 5565-69; Fox et al., (1981) Dev. Biol. 83, 391-98; Bird and Kimber, (1984) Dev. Biol. 104, 449-60; Muramatsu, (1994) Nagoya J. Med. Sci. 57, 95-108; Marani et al., (1986) Acta. Morphol. Neerl. Scand. 24, 103-110; Gomperts et al., (1994) Development 120, 135-41). Intriguingly, CNS cell sub-populations in various species also express this marker during development and in adulthood. LeX is expressed in germinal zones in the murine embryonic telencephalon (Yamamoto et al., (1985) Proc. Natl. Acad. Sci. USA 82, 3045-49; Allendoerfer et al., (1995) Mol. Cell. Neurosci. 6, 381-95; Allendoerfer et al., (1999) Dev. Biol. 211, 208-19; Tole et al., (1995) J. Neurosci 15, 624-27; Ashwell and Mai, (1997) Cell Tissue Res. 289, 17-23) and spinal cord (Dodd and Jessell, (1986) J. Exp. Biol. 129, 225-38), and in the cerebellar external granular layer (Marani and Tetteroo, (1983) Histochemistry 78, 157-61. In the adult mouse CNS, LeX is expressed by sub-populations of astrocytes, tanycytes, and a few neurons (Bartsch and Mai, (1991) Cell Tissue Res. 263, 353-66; Gocht et al., (1996) Histol. Histopathol. 11, 1007-28; Ashwell and Mai, (1997) Cell Tissue Res. 289, 17-23).

TABLE B
SEQUENCE OF CD15 (SEQ ID NO:2)

| | |
|---|---|
| 1 | ctgctcctgc gcggcagctg ctttagaagg tctcgagcct cctgtacctt cccagggatg |
| 61 | aaccgggcct tccctctgga aggcgagggt tcgggccaca gtgagcgagg gccagggcgg |
| 121 | tgggcgcgcg cagagggaaa ccggatcagt tgagagagaa tcaagagtag cggatgaggc |
| 181 | gcttgtgggg cgcggcccgg aagccctcgg gcgcgggctg ggagaaggag tgggcggagg |
| 241 | cgccgcagga ggctcccggg gcctggtcgg gccggctggg ccccgggcgc agtggaagaa |
| 301 | agggacgggc ggtgcccggt tgggcgtcct ggccagctca ccttgccctg gcggctcgcc |
| 361 | ccgcccggca cttgggagga gcagggcagg gcccgcggcc tttgcattct gggaccgccc |
| 421 | ccttccattc ccgggccagc ggcgagcggc agcgacggct ggagccgcag ctacagcatg |
| 481 | agagccggtg ccgctcctcc acgcctgcgg acgcgtggcg agcggaggca gcgctgcctg |
| 541 | ttcgcgccat gggggcaccg tggggctcgc cgacggcggc ggcgggcggg cggcgcgggt |
| 601 | ggcgccgagg ccgggggctg ccatggaccg tctgtgtgct ggcggccgcc ggcttgacgt |
| 661 | gtacggcgct gatcacctac gcttgctggg ggcagctgcc gccgctgccc tgggcgtcgc |
| 721 | caaccccgtc gcgaccggtg ggcgtgctgc tgtggtggga gcccttcggg gggcgcgata |
| 781 | gcgccccgag gccgccccct gactgccggc tgcgcttcaa catcagcggc tgccgcctgc |
| 841 | tcaccgaccg cgcgtcctac ggagaggctc aggccgtgct tttccaccac cgcgacctcg |
| 901 | tgaaggggcc ccccgactgg cccccgccct ggggcatcca ggcgcacact gccgaggagg |
| 961 | tggatctgcg cgtgttggac tacgaggagg cagcggcggc ggcagaagcc ctggcgacct |
| 1021 | ccagccccag gcccccgggc cagcgctggg tttggatgaa cttcgagtcg ccctcgcact |
| 1081 | ccccggggct gcgaagcctg gcaagtaacc tcttcaactg gacgctctcc taccgggcgg |
| 1141 | actcggacgt ctttgtgcct tatggctacc tctaccccag aagccacccc ggcgacccgc |
| 1201 | cctcaggcct ggccccgcca ctgtccagga aacaggggct ggtggcatgg gtggtgagcc |
| 1261 | actgggacga gcgccaggcc cgggtccgct actaccacca actgagccaa catgtgaccg |
| 1321 | tggacgtgtt cggccggggc gggccggggc agccggtgcc cgaaattggg ctcctgcaca |
| 1381 | cagtggcccg ctacaagttc tacctggctt tcgagaactc gcagcacctg gattatatca |
| 1441 | ccgagaagct ctggcgcaac gcgttgctcg ctggggcggt gccggtggtg ctgggcccag |
| 1501 | accgtgccaa ctacgagcgc tttgtgcccc gcggcgcctt catccacgtg gacgacttcc |
| 1561 | caagtgcctc ctccctggcc tcgtacctgc ttttcctcga ccgcaacccc gcggtctatc |
| 1621 | gccgctactt ccactggcgc cggagctacg ctgtccacat cacctccttc tgggacgagc |
| 1681 | cttggtgccg ggtgtgccag gctgtacaga gggctgggga ccggcccaag agcatacgga |
| 1741 | acttggccag ctggttcgag cggtgaagcc gcgctcccct ggaagcgacc caggggaggc |
| 1801 | caagttgtca gctttttgat cctctactgt gcatctcctt gactgccgca tcatgggagt |
| 1861 | aagttcttca aacacccatt tttgctctat gggaaaaaaa cgatttacca attaatatta |
| 1921 | ctcagcacag agatgggggc ccggtttcca tattttttgc acagctagca attgggctcc |
| 1981 | ctttgctgct gatgggcatc attgtttagg ggtgaaggag ggggttcttc ctcaccttgt |
| 2041 | aaccagtgca gaaatgaaat agcttagcgg caagaagccg ttgaggcggt ttcctgaatt |
| 2101 | tccccatctg ccacaggcca tatttgtggc ccgtgcagct tccaaatctc atacacaact |
| 2161 | gttcccgatt cacgtttttc tggaccaagg tgaagcaaat ttgtggttgt agaaggagcc |
| 2221 | ttgttggtgg agagtggaag gactgtggct gcaggtggga ctttgttgtt tggattcctc |
| 2281 | acagccttgg ctcctgagaa aggtgaggag ggcagtccaa gaggggccgc tgacttcttt |
| 2341 | cacaagtact atctgttccc ctgtcctgtg aatggaagca aagtgctgga ttgtccttgg |
| 2401 | aggaaactta agatgaatac atgcgtgtac ctcactttac ataagaaatg tattcctgaa |
| 2461 | aagctgcatt taaatcaagt cccaaattca ttgacttagg ggagttcagt atttaatgaa |
| 2521 | accctatgga gaatttatcc ctttacaatg tgaatagtca tctcctaatt tgtttcttct |
| 2581 | gtctttatgt ttttctataa cctggatttt ttaaatcata ttaaaattac agatgtgaaa |
| 2641 | ataaagcaga agcaaccttt ttccctcttc ccagaaaacc agtctgtgtt tacagacaga |
| 2701 | agagaaggaa gccatagtgt cacttccaca caattattta tttcatgtct ttactggacc |
| 2761 | tgaaatttaa actgcaatgc cagtcctgca ggagtgctgg cattaccctc tgcagaacag |
| 2821 | tgaaaggtat tgcactacat tatggaatca tgcaaaaaaa a |

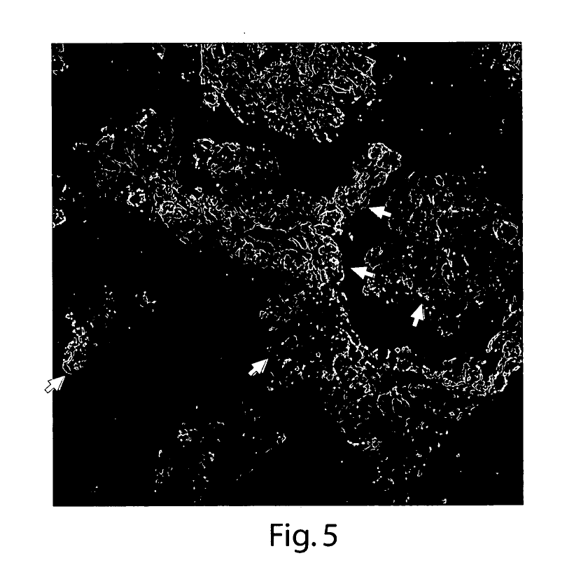

CD15⁺ cells are present in the pancreas. In most cases, the CD15 antigen is expressed on endocrine cells, presumably glucagon-producing cells. See FIG. 5. Most pancreatic ductal cells do not express CD15, however, when a duct has an opening hole, the luminal surface stains for CD15 antigen. Unlike CD133⁺ expression, CD15 is not observed on closed ducts. Pancreatic stem cells are CD15⁻. In contrast, there are several populations of pancreatic progenitor cells. Some pancreatic progenitor cells are CD15⁺ and some are CD15⁻.

CD9 antigen is a 24 to 27 kDa glycoprotein expressed on the surface of developing B lymphocytes, platelets, monocytes, eosinophils, basophils, stimulated T lymphocytes and by neurons and glial cells in the peripheral nervous system. It belongs to a family of membrane proteins termed tetraspanins which transverse the membrane four times. In pre-B cells and platelets, CD9 antigen regulates cell activation and aggregation possibly through an association with the integrin CD41/CD61 (GPIIb/GPIIIa). It also regulates cell motility in a variety of cell lines, and appears to be an important regulator of Schwann cell behavior in peripheral nerve. In melanoma and breast cancer, CD9 antigen expression may indicate a favorable prognosis as expression has been shown to occur predominantly on primary, non-metastatic tumors.

Antibodies that recognize CD9 are commercially available from Research Diagnostics Inc., Flanders, N.J. e.g., CLB-trom/8, 4E1 (catalog numbers RDI-M1362clb and RDI-M1666clb), MM2/57 (catalog numbers RDI-CBL162, RDI-CBL162FT, and RDI-CBL162PE), BU16 (catalog number RDI-CBL560), and 72F6 (catalog number RDI-CD9abm-72).

Isolation of Subsets of Stem and Progenitor Cells

Establishing a hierarchy of a particular cell fate map has now been accomplished for the mouse hematopoietic stem cells and its progeny. This fate mapping uses the techniques that have been applied in this invention and can be found more descriptively in Morrison et al., Immunity 1994 November; 1(8):661-73; Kondo M et al., Cell 1997 Nov. 28;91(5):661-72; Akashi et al., Nature 2000 Mar. 9;404(6774):193-7. The further dissection of the initially described mouse hematopoietic stem cell population was accomplished by using surface phenotypes to subdivide the hematopoietic stem cell population into both a short and long term repopulating fraction. This technology was then applied to the progeny of the hematopoietic stem cells to identify a lymphomyeloid progenitor; a myeloid restricted progenitor, and a common lymphoid progenitor.

Isolation of Pancreatic Stem Cells and Progenitor Cells

The invention provides for positive selection methodologies using the cellular markers CD49f and CD133 that can be used to isolate subsets of pancreatic cells or gastrointestinal cells, including stem cells and progenitors. The isolated subsets include CD49f⁺ cells and CD133⁺ cells. The invention also provides for positive selection methodologies using the cellular markers CD9 and CD15 that can be used to isolate subsets of pancreatic progenitor cells. The invention also provides for negative selection methodologies using the cellular markers CD34 and CD45 that can be used to isolate subsets of pancreatic cells or gastrointestinal cells. The isolated subsets include CD34⁻ and CD45⁻ cells. The invention also provides for negative selection methodologies using the cellular markers CD9 and CD15 that can be used to isolate pancreatic stem cells. Isolation of such subsets can be performed either individually or in combination, including sequentially (in any order).

Cell Deposits. As noted in U.S. Pat. No. 5,843,633, the murine hybridoma cell line AC133 was deposited at the American Type Tissue Collection, 12301 Parklawn Drive, Rockville, Md. 20852 (ATCC designation HB12346) in accordance with the provisions of the Budapest Treaty. The SC111 antibody was deposited at the ATCC in accordance with the provisions of the Budapest Treaty on ______ (ATCC designation ______).

Isolation, enrichment, and selection of cells. The population of cells from which pancreatic stem cells or progenitor cells or gastrointestinal stem cells or progenitor cells are isolated can be a pancreatic tissue, a population of cells dissociated from pancreatic tissue, islets of Langerhans, pancreatic-derived cells, gastrointestinal tissue, a population of cells dissociated from gastrointestinal tissue, gastrointestinal-derived cells or gastrointestinal-derived explants. The cells can be obtained from fetal, neonatal, juvenile, adult, or postmortem tissues. The cells can be cultured in vitro, e.g., a suspension culture or an adherent culture.

The invention provides for the isolation and identification of pancreatic stem cells and/or progenitors. Identification of a pancreatic stem cells or progenitors involves contacting a population of pancreatic cells, pancreatic-derived cells, gastrointestinal cells, or gastrointestinal-derived cells with a reagent that binds to the CD49f antigen and/or a reagent that binds to the CD133 antigen, and detecting the contact between the reagent that binds to the CD49f and/or CD133 antigens and the CD49f and/or CD133 antigens on the surface of cells. Those cells to which the CD49f and/or CD133 reagents bind are identified as pancreatic stem cells and/or progenitors. The identity of these cells can be confirmed by assays that demonstrate whether the cells are in fact pancreatic stem cells, capable of self-renewal and multipotency. Identification of pancreatic stem cells or progenitors also involves contacting a population of pancreatic cells, pancreatic-derived cells, gastrointestinal cells, or gastrointestinal-derived cells with a reagent that binds to the CD34 antigen and/or a reagent that binds to the CD45 antigen and detecting the contact between the reagent that binds to the CD34 and/or CD45 antigens and the CD34 and/or CD45 antigens on the surface of cells. Those cells which do not bind the CD34 and/or CD45 reagents are identified as or are enriched for pancreatic stem cells and/or progenitors. The identity of these cells can be confirmed by assays that demonstrate whether the cells are in fact pancreatic stem cells, capable of self-renewal and multipotency.

The methods of this invention can also be used to isolate CD49f⁺ cells from CD49f^{-/lo} cells using an anti-CD49f antibody, or CD133⁺ cells from CD133⁺ cells using an anti-CD133^{-/lo} antibody, by a positive selection technique of combining a population of pancreas cells which contains a fraction of pancreatic stem cells and/or progenitors with a reagent that specifically binds to the CD49f antigen or the CD133 antigen, and then selecting for CD49f⁺ cells or CD133⁺ cells to produce a selected population enriched in CD49f⁺ and/or CD133⁺ pancreatic stem cells and/or progenitors as compared with the population of pancreatic cells or gastrointestinal cells prior to the selection. Methods are also provided to isolate or enrich for CD49f⁺ cells from CD49f^{-/lo} cells or CD133⁺ cells from CD133^{-/lo} cells using an anti-CD34 antibody and/or an anti-CD45 antibody, by a negative selection technique of combining a population of pancreas cells which contains a fraction of pancreatic stem cells and/or progenitors with a reagent that specifically binds to the CD34 antigen or the CD45 antigen, and removing the CD34⁺ and/or CD45⁺ cells to produce a selected population enriched in CD49f⁺ and/or CD133⁺ pancreatic stem cells and/or progenitors as compared with the population of pancreatic cells or gastrointestinal cells prior to the selection.

Accordingly, the invention further provides for the enrichment of pancreatic stem cells and/or progenitors from pancreatic tissue or gastrointestinal tissue or gastrointestinal-derived explants or pancreatic stem cell cultures (e.g., suspension cultures or adherent cultures). The invention is thus useful for the enrichment of pancreatic stem cells and/or progenitors from pancreatic tissue or gastrointestinal tissue or gastrointestinal-derived explants in which stem cells and progenitor cells occur at low frequency, or may have been depleted, such as late embryo, fetal, neonatal, juvenile, adult and/or postmortem tissue. One of ordinary skill in the art can combine a population of pancreatic cells or gastrointestinal cells containing a fraction of pancreatic stem cells and/or progenitors with a reagent that specifically binds to the CD49f antigen or to the CD133 antigen, and select for the CD49f⁺ or CD133⁺ cells. In this way, the selected CD49f⁺ or CD133⁺ cells are enriched in the fraction of pancreatic stem cells and/or progenitors as compared with the population of pancreatic cells or gastrointestinal cells.

The invention also provides an antibody that specifically binds to the CD49f antigen, wherein the CD49f antigen specifically binds to the GoH3 and/or 4F10 antibodies. This antibody may be produced by a hybridoma cell line. This monoclonal antibody may block simultaneous binding to the CD49f antigen by the antibody GoH3 and/or the antibody 4F10. Of particular interest are antibodies that bind to the CD49f antigen, cross-reactive antibodies (i.e., those which bind to the same epitope as the GoH3 and/or 4F10 antibodies and substantially inhibit simultaneous binding), species analogs thereof, binding fragments thereof, and conjugates thereof.

Similarly, the invention also provides an antibody that specifically binds to the CD133 antigen, wherein the CD133 antigen specifically binds to the AC133 and/or SC111 antibodies. This antibody may be produced by a hybridoma cell line. This monoclonal antibody may block simultaneous binding to the CD133 antigen by the antibody AC133 and/or the antibody SC111. Of particular interest are antibodies that bind to the CD133 antigen, cross-reactive antibodies (i.e., those which bind to the same epitope as the AC133 and/or SC111 antibodies and substantially inhibit simultaneous binding), species analogs thereof, binding fragments thereof, and conjugates thereof.

Likewise, the invention also provides an antibody that specifically binds to the CD15 antigen, wherein the CD15 antigen specifically binds to the MMA antibody. This antibody may be produced by a hybridoma cell line. This monoclonal antibody may block simultaneous binding to the CD15 antigen by the antibody MMA. Of particular interest are antibodies that bind to the CD15 antigen, cross-reactive antibodies (i.e., those which bind to the same epitope as the MMA antibody and substantially inhibit simultaneous binding), species analogs thereof, binding fragments thereof, and conjugates thereof.

Also provided are methods for the further enrichment of human pancreatic stem cells and/or progenitors by combining a population of CD49f⁺ or a population of CD133⁺ pancreatic cells, pancreatic-derived cells, gastrointestinal cells, or gastrointestinal-derived cells with a reagent that specifically binds to the CD34 antigen or CD45 antigen and removing those cells that bind to the CD34 antigen or CD45 antigen, wherein the remaining cells are enriched for pancreatic stem cells and/or progenitors. In some embodiments, this reagent is an antibody. Also included are methods for the further enrichment of human pancreatic stem cells and/or progenitors by combining a population of CD34⁻ or a population of CD45⁻ pancreatic cells, pancreatic-derived cells, gastrointestinal cells, or gastrointestinal-derived cells with a reagent that specifically binds to the CD49f antigen or CD133 antigen and selecting for those cells that bind to the CD49f antigen or CD133 antigen, wherein the selected cells are enriched for pancreatic stem cells and/or progenitors.

In any of the methods of this invention, the population of pancreatic cells, pancreatic-derived cells, gastrointestinal cells, or gastrointestinal-derived cells can be enriched by contacting the cells with a reagent that specifically binds to the CD133 antigen before, during, and/or after contacting the cells with a reagent that binds to the CD49f antigen. Likewise, in any of the methods of this invention, the population of pancreatic cells, pancreatic-derived cells, gastrointestinal cells, or gastrointestinal-derived cells can be enriched by contacting the cells with a reagent that specifically binds to the CD49f antigen before, during, and/or after contacting the cells with a reagent that binds to the CD133 antigen.

Cell selection according to the invention can be accomplished by any suitable means known in the art, including flow cytometry, such as by fluorescence activated cell sorting using fluorochrome conjugated antibodies. The selection can also be by high gradient magnetic selection using antibodies conjugated to magnetic particles. Any other suitable method including attachment to and disattachment from solid phase, is also contemplated within the scope of the invention.

A population of cells can be derived by immunoselection using an anti-CD49f antibody, or an anti-CD133 antibody. The population of cells should contain at least 30% CD49f⁺ and/or CD133⁺ pancreatic stem cells or progenitor cells, preferably at least 50-70% CD49f⁺ and/or CD133⁺ pancreatic stem cells or progenitor cells, and more preferably greater than 90% CD49f⁺ and/or CD133⁺ pancreatic stem cells or progenitor cells. Most preferable would be a substantially pure population of CD49f⁺ and/or CD133⁺ pancreatic stem cells or progenitor cells, containing at least 95% CD49f⁺ and/or CD133⁺ pancreatic stem cells or progenitor cells. The degree of enrichment obtained, and actually used, depends on a number of factors, including e.g., the method of selection, the method of growth, and the cell dose of the cells that are placed in culture.

The population of cells can be derived from late embryo, fetal, neonatal, juvenile, adult, and/or postmortem mammalian pancreas tissue or gastrointestinal tissue or gastrointestinal-derived explants. In the most preferred embodiment, the pancreatic stem cells or progenitor cells are human. In some embodiments, the CD49f⁺ and/or CD133⁺ cells in the population can be complexed to endothelial cells.

The in vitro cell cultures described herein containing an enriched population of pancreatic stem cells or progenitor cells or other cells may be characterized as producing progeny that stain positive for insulin, glucagons, or somatostatin and, in the presence of differentiation-inducing conditions, produce progeny cells that differentiate into mature pancreatic cells, including β islet cells, α islet cells, δ islet cells, or φ islet cells (PP cells), especially glucose-responsive, insulin-secreting cells and glucagon-producing cells.

One skilled in the art can introduce an isolated CD49f⁺ and/or CD133⁺ cell to a culture medium, proliferate the isolated CD49f⁺ and/or CD133⁺ cell in culture; culture the progeny of the isolated CD49f⁺ and/or CD133⁺ cell under conditions in which the isolated CD49f⁺ and/or CD133⁺ cell differentiates to mature pancreatic cells; then detect the presence of mature pancreatic cells, including β islet cells, α islet cells, δ islet cells, or φ islet cells (PP cells). The presence of multiple types of mature pancreatic cells characterizes the isolated CD49f⁺ and/or CD133⁺ cell as a pancreatic stem cell.

The culture in which the isolated CD49f⁺ and/or CD133⁺ cell proliferates can be a serum-free medium containing one or more predetermined growth factors effective for inducing multipotent pancreatic stem cell proliferation. The culture medium can be supplemented with a growth factor. The conditions in which the CD49f⁺ and/or CD133⁺ cell differentiates to β islet cells, α islet cells, δ islet cells, or φ islet cells (PP cells) can include culturing the CD49f⁺ and/or CD133⁺ cell progeny on a surface in culture medium containing a growth factor.

The invention also provides a method for identifying the presence of a growth factor that affects the growth of pancreatic stem cells or progenitor cells. One skilled in the art can combine a composition suspected of containing at least one growth factor that affects the growth of pancreatic stem cells or progenitor cells with a composition containing pancreatic stem cells or progenitor cells, then determine the growth of the pancreatic stem cells or progenitor cells as a function of the presence of the composition. Altered (e.g., increased, decreased, etc.) pancreatic stem cells or progenitor cell growth indicates the presence in the composition of a growth factor that affects the growth of pancreatic stem cells or progenitor cells. The identity of the growth factor can be determined using techniques known in the art.

Antibodies to CD133. Antibodies to CD133 may be obtained or prepared as discussed in U.S. Pat. No. 5,843,633, incorporated herein by reference. The CD133 antigen can be contacted with an antibody, such as various anti-CD133 monoclonal antibodies, including but not limited to AC133 and SC111, which have specificity for the CD133 antigen. “Anti-CD133 antibodies” are characterized by binding to the CD133 protein in native, in FACS and immunoprecipitation experiments, as well as denatured, in western blot experiments, conformation. The CD133 antigen has been reported to have several reduced molecular weights in the range of 125 kDa to 127 kDa according to U.S. Pat. No. 5,843,633 and 115 kDa to 127 kDa according to United States Published Patent Application No. 20010051372.

Antibodies to CD49f Antibodies to CD49f may be obtained commercially or prepared according to methods known to those of ordinary skill in the art. The CD49f antigen can be contacted with an antibody, such as various anti-CD49f monoclonal antibodies, which have specificity for the CD49f antigen. Anti-CD49f antibodies are characterized by binding to the CD49f antigen under Western blot conditions from reducing SDS-PAGE gels. As used herein, the term “anti-CD49f antibody” refers to a monoclonal or polyclonal antibody that specifically binds to the CD49f antigen. Examples of anti-CD49f antibodies include, but are not limited to, GoH3 and 4F10. The CD49f antigen has a molecular weight, based on commercially available standards, in the range of about 140 kDa. The CD49f antigen is expressed on thymocytes, T lymphocytes, and monocytes. Increased expression is found on activated and memory T cells. The A splice variant alone is expressed in the lung, liver, spleen and cervix. The B splice variant alone is expressed in the brain, ovary, and kidney. Both forms are also detected in other tissues.

Antibodies to CD15. Antibodies to human CD15 may be obtained commercially or prepared according to methods known to those of ordinary skill in the art. The CD15 antigen can be contacted with an antibody, such as various anti-CD15 monoclonal antibodies, which have specificity for the CD15 antigen. Anti-CD15 antibodies are characterized by binding to the CD15 antigen under Western blot conditions from reducing SDS-PAGE gels. As used herein, the term “anti-CD15 antibody” refers to a monoclonal or polyclonal antibody that specifically binds to the CD15 antigen. Examples of anti-CD15 antibodies include, but are not limited to, MMA. The CD15 antigen has a molecular weight, based on commercially available standards, in the range of about 220 kDa. The CD15 antigen is expressed in mature human neutrophils, monocytes, and eosinophils. It can also be found present on embryonic tissues and adenocarcinomas, myeloid leukemias and Reed-Sternberg cells.

Antibodies to the CD133, CD49f, CD15, CD9, CD34, and/or CD45 antigens can be obtained by immunizing a xenogeneic immunocompetent mammalian host (including murine, rodentia, lagomorpha, ovine, porcine, bovine, etc.) with human progenitor cells. The choice of a particular host is primarily one of convenience. A suitable progenitor cell population for immunization can be obtained from pancreatic stem cells. Immunizations are performed in accordance with conventional techniques, where the cells may be injected subcutaneously, intramuscularly, intraperitoneally, intravascularly, etc. Normally, from about 10⁶ to 10⁸ cells are used, which may be divided into one or more injections, usually not more than about 8 injections, over a period of from about one to about three weeks. The injections may be with or without adjuvant, e.g. complete or incomplete Freund's adjuvant, specol, alum, etc.

After completion of the immunization schedule, the antiserum may be harvested in accordance with conventional methods to provide polygonal antisera specific for the surface membrane proteins of progenitor cells, including the CD133, CD49f, CD15, CD9, CD34, and/or CD45 antigens. Lymphocytes are harvested from the appropriate lymphoid tissue, e.g. spleen, draining lymph node, etc., and fused with an appropriate fusion partner, usually a myeloma line, producing a hybridoma secreting a specific monoclonal antibody. Screening clones of hybridomas for the antigenic specificity of interest is performed in accordance with conventional methods.

The anti-CD133, anti-CD49f, anti-CD15, anti-CD9, anti-CD34, and/or anti-CD45 antibodies can be produced as a single chain, instead of the normal multimeric structure. Single chain antibodies are described in e.g., Jost et al., 269 J. BIOL. CHEM. 26267-73 (1994), incorporated herein by reference. DNA sequences encoding the variable region of the heavy chain and the variable region of the light chain are ligated to a spacer encoding at least about 4 amino acids of small neutral amino acids, including glycine or serine. The protein encoded by this fusion allows assembly of a functional variable region that retains the specificity and affinity of the original antibody. Anti-CD133, anti-C49f, anti-CD15, anti-CD9, anti-CD34, and/or anti-CD45 antibodies can also be produced by use of Ig cDNA for construction of chimeric immunoglobulin genes (Liu et al., 84 PROC. NATL. ACAD. SCI. 3439 (1987) and 139 J. IMMUNOL. 3521 (1987), incorporated herein by reference. mRNA is isolated from a hybridoma or other cell producing the antibody and used to produce cDNA. The cDNA of interest may be amplified by the polymerase chain reaction using specific primers (See U.S. Pat. No. 4,683,195 and U.S. Pat. No. 4,683,202).

Alternatively, a library is made and screened to isolate the sequence of interest. The DNA sequence encoding the variable region of the antibody is then fused to human constant region sequences. The sequences of human constant regions genes may be found in Kabat et al., N.I.H. PUBLICATION No. 91-3242 (1991). Human C region genes are readily available from known clones. The chimeric, humanized antibody is then expressed by conventional methods.

Anti-CD133, anti-CD49f, anti-CD15, anti-CD9, anti-CD34, and/or anti-CD45 antibodies can also be produced as antibody fragments, such as Fv, F(ab′)₂ and Fab. Antibody fragments may be prepared by cleavage of the intact protein, e.g. by protease or chemical cleavage. Alternatively, a truncated gene can be designed. For example, a chimeric gene encoding a portion of the F(ab′)₂ fragment would include DNA sequences encoding the CH1 domain and hinge region of the H chain, followed by a translational stop codon to yield the truncated molecule.

Immunostaining. Biological samples are assayed for the presence of CD133⁺, CD49f, CD15+, CD9+, CD34⁺, and/or CD45⁺ cells by any convenient immunoassay method for the presence of cells expressing the surface molecule bound by the subject antibodies. Assays may be performed on cell lysates, intact cells, frozen sections, etc. Any commercially available antibodies are suitable for the direct immunofluorescent staining of cells.

Cell sorting. The use of cell surface antigens found on pancreatic stem cells and progenitor cells provides a means for the positive immunoselection of stem cell or progenitor cell populations, as well as for the phenotypic analysis of stem cell or progenitor cell populations using flow cytometry. Cells selected for expression of CD49f and/or CD133 antigen may be further purified by selection for other stem cell and progenitor cell markers. For example, progenitor cells can be further subdivided by positive immunoselection for CD9 or CD15. For the preparation of substantially pure progenitors and stem cells, a subset of progenitor cells is separated from other cells on the basis of CD49f and/or CD133 binding. Selection with these markers can be accomplished in any combination and/or in any order. Procedures for separation may include magnetic separation, using antibody-coated magnetic beads, affinity chromatography and “panning” with antibody attached to a solid matrix, e.g. a plate, or other convenient technique. Techniques providing accurate separation include fluorescence activated cell sorters, which can have varying degrees of sophistication, such as multiple color channels, low angle and obtuse light scattering detecting channels, impedance channels, etc. Dead cells may be eliminated by selection with dyes associated with dead cells (e.g. propidium iodide [PI], LDS). Any technique known to those in the art, which is not unduly detrimental to the viability of the selected cells, may be employed.

Conveniently, the antibodies are conjugated with labels to allow for ease of separation of the particular cell type, e.g. magnetic beads; biotin, which binds with high affinity to avidin or streptavidin; fluorochromes, which can be used with a fluorescence activated cell sorter; haptens; and the like. Multi-color analyses may be employed with the FACS or in a combination of immunomagnetic separation and flow cytometry. Multi-color analysis is of interest for the separation of cells based on multiple surface antigens, e.g. CD133⁺CD49f⁺, CD133⁺CD49f⁺CD34⁻CD45⁻; CD133⁺CD49f⁺CD34⁻, CD133⁺CD49f⁺CD45⁻, CD49f⁺CD34⁻, CD49f⁺CD45⁻, CD133⁺CD34⁻, CD133⁺CD45⁻, CD34⁻CD45⁻, CD133⁻CD49f⁻CD34⁺, CD133⁻CD49f⁻, CD45⁺, CD34⁺CD45⁺, CD133⁻CD49f⁻CD34⁺CD45⁺, CD133⁺CD49f⁺CD9⁻CD15₋, CD9⁻CD15⁻, CD133⁺CD49f⁺CD9⁻CD15+, CD133⁺CD49f⁺CD9^{+CD}15⁻, etc. Fluorochromes which find use in a multi-color analysis include phycobiliproteins, e.g. phycoerythrin and allophycocyanins; fluorescein and Texas red. A negative designation indicates that the level of staining is at or below the brightness of an isotype matched negative control. A dim or low designation indicates that the level of staining may be near the level of a negative stain, but may also be brighter than an isotype matched control.

In one embodiment, the antibodies are directly or indirectly conjugated to a magnetic reagent, such as a superparamagnetic microparticle (microparticle). Direct conjugation to a microparticle can be achieved by use of various chemical linking groups, as known in the art. The antibody can be coupled to the microparticles through side chain amino or sulfhydryl groups and heterofunctional cross-linking reagents. A large number of heterofunctional compounds are available for linking to entities. A preferred linking group is 3-(2-pyridyidithio)propionic acid N-hydroxysuccinimide ester (SPDP) or 4-(N-maleimidomethyl)-cyclohexane-1-carboxylic acid N-hydroxysuccinimide ester (SMCC) with a reactive sulfhydryl group on the antibody and a reactive amino group on the magnetic particle.

Alternatively, the antibodies can be indirectly coupled to the magnetic particles. The antibody is directly conjugated to a hapten, and hapten-specific, second stage antibodies are conjugated to the particles. Suitable haptens include digoxin, digoxigenin, FITC, dinitrophenyl, nitrophenyl, avidin, biotin, etc. Methods for conjugation of the hapten to a protein are known in the art, and kits for such conjugations are commercially available.

To practice the methods of the invention, the antibodies are added to a cell sample. The amount of antibody necessary to bind a particular cell subset is empirically determined by performing a test separation and analysis. The cells and antibodies are incubated for a period of time sufficient for complexes to form, usually at least about 5 minutes, more usually at least about 10 minutes, and usually not more than one hour, more usually not more than about 30 minutes.

The cells may additionally be incubated with antibodies or binding molecules specific for cell surface markers known to be present or absent on progenitor or stem cells. The labeled cells are separated in accordance with the specific antibody preparation. Fluorochrome labeled antibodies are useful for FACS separation, magnetic particles for immunomagnetic selection, particularly high gradient magnetic selection (HGMS), etc. Exemplary magnetic separation devices are described in, e.g. WO 90/07380, PCT/US96/00953, and EP 438,520. The AC133 Cell Isolation Kit (Miltenyi Biotec Inc., Auburn Calif.) can be used for the positive selection of AC133⁺ cells. The kit provides a tool for single step isolation of AC133⁺ cells (i.e., cells that have the CD133 antigen). The AC133 Cell Isolation Kit contains FcR Blocking Reagent and MACS colloidal MicroBeads conjugated to the monoclonal mouse anti-human AC133 antibody.

The purified cell population may be collected in any appropriate medium. Various commercially available media may be used, including Dulbecco's Modified Eagle Medium (DMEM), Hank's Basic Salt Solution (HBSS), Dulbecco's phosphate buffered saline (DPBS), RPMI, Iscove's modified Dulbecco's medium (IMDM), phosphate buffered saline (PBS) with 5 mM EDTA, etc., frequently supplemented with fetal calf serum (FCS), bovine serum albumin (BSA), human serum albumin (HSA), etc.

Populations highly enriched for human progenitor or stem cells are achieved in this manner. The desired cells will be 30% or more of the cell composition, preferably 50% or more of the cell population, more preferably 90% or more of the cell population, and most preferably 95% or more (e.g. substantially pure) of the cell population.

Use of purified stem cell/progenitor cells. CD133⁺CD49f⁺, CD133⁺CD49f⁺CD34⁻, CD133⁺CD49f⁺CD45⁻, CD133⁺CD49f⁺CD34⁻CD45⁻, CD133⁺CD49f⁺CD9⁻CD15⁻, CD133⁺CD49f⁺CD9⁻CD15⁺, CD133⁺CD49f⁺CD9⁺CD15⁻ stem cells or progenitor cells are useful in a variety of ways. The CD133⁺CD49f⁺, CD133⁺CD49f⁺CD34⁻, CD133⁺CD49f⁺CD45⁻, CD133⁺CD49f⁺CD34⁻CD45⁻, CD133⁺CD49f⁺CD9⁻CD15⁻, CD133⁺CD49f⁻CD9⁻CD15⁺, CD133⁺CD49f⁺CD9⁺CD15⁻ cells can be used to reconstitute a host whose cells have been lost through disease or injury. Genetic diseases associated with cells may be treated by genetic modification of autologous or allogeneic stem cells to correct a genetic defect or treat to protect against disease. Alternatively, normal allogeneic progenitor cells may be transplanted. Diseases other than those associated with cells may also be treated, where the disease is related to the lack of a particular secreted product such as hormone, enzyme, growth factor, or the like.

The isolated cells of the invention can be used in the treatment of a variety of pancreatic disorders, both exocrine and endocrine. For instance, the stem cells or progenitor cells can be used to produce populations of differentiated pancreatic cells for repair subsequent to partial pancreatectomy, e.g., excision of a portion of the pancreas. Likewise, such cell populations can be used to regenerate or replace pancreatic tissue loss due to, pancreatolysis, e.g., destruction of pancreatic tissue, such as pancreatitis, e.g., a condition due to autolysis of pancreatic tissue caused by escape of enzymes into the substance.

The isolated pancreatic stem cells or progenitor cells can be provided for patients suffering from any insulin-deficiency disorder, including diabetes. Diabetes is characterized by pancreatic islet destruction or dysfunction leading to loss of glucose control. Diabetes mellitus is a metabolic disorder defined by the presence of chronically elevated levels of blood glucose (hyperglycemia). Insulin-dependent (Type 1) diabetes mellitus (“IDDM”) results from an autoimmune-mediated destruction of the pancreatic β-cells with consequent loss of insulin production, which results in hyperglycemia. Type 1 diabetics require insulin replacement therapy to ensure survival. Non-insulin-dependent (Type 2) diabetes mellitus (“NIDDM”) is initially characterized by hyperglycemia in the presence of higher-than-normal levels of plasma insulin (hyperinsulinemia). In Type 2 diabetes, tissue processes which control carbohydrate metabolism are believed to have decreased sensitivity to insulin. Progression of the Type 2 diabetic state is associated with increasing concentrations of blood glucose, and coupled with a relative decrease in the rate of glucose-induced insulin secretion.

The pancreatic stem cells or progenitor cells of the invention can be used for treatment of diabetes because they have the ability to differentiate into cells of pancreatic lineage, e.g., p islet cells. The progenitor cells of the invention can be cultured in vitro under conditions which can further induce these cells to differentiate into mature pancreatic cells, or they can undergo differentiation in vivo once introduced into a subject.

In addition to providing a source of implantable cells, either in the form of the stem cell population, the progenitor cell population or the differentiated progeny thereof, the subject cells can be used to produce cultures of pancreatic cells for the production and purification of secreted factors. For instance, cultured cells can be provided as a source of insulin. Likewise, exocrine cultures can be provided as a source for pancreatin.

The CD133⁺CD49f⁺, CD133⁺CD49f⁺CD9⁻CD15⁻, CD133⁺CD49f⁺CD9-CD15⁺ and/or CD133⁺CD49f⁺CD9⁺CD15⁻ cells may also be used in the isolation and evaluation of factors associated with the differentiation and maturation of cells. Thus, the cells may be used in assays to determine the activity of media, such as conditioned media; to evaluate fluids for growth factor activity, involvement with dedication of lineages, or the like.

The CD133⁺CD49f⁺, CD133⁺CD49f⁺CD9⁻CD15⁻, CD133⁺CD49f⁺CD9⁻CD15⁺ and/or CD133⁺CD49f⁺CD9⁺CD15⁻ cells may be frozen at liquid nitrogen temperatures and stored for long periods of time, being thawed and capable of being reused. The cells will usually be stored in 5% DMSO and 95% fetal calf serum. Once thawed, the cells may be expanded by use of growth factors or stromal cells associated with stem cell proliferation and differentiation.

### Other Embodiments

It is to be understood that, while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of producing a population from pancreatic tissue enriched for human pancreatic stem cells, progenitors, or a combination thereof, comprising:
a) contacting pancreatic tissue, pancreatic cells, pancreatic-derived cells, or primary gastrointestinal tissue or gastrointestinal-derived cells with a monoclonal antibody that binds CD133 or CD49f; and
b) selecting said pancreatic tissue, pancreatic cells, pancreatic-derived cells, or primary gastrointestinal tissue or gastrointestinal-derived cells that bind to the monoclonal antibody;
wherein the selected cells are enriched for human pancreatic stem cells, progenitors, or a combination thereof.

2. The method of claim 1, wherein the population containing pancreatic stem/progenitor cells, is obtained from a suspension culture or an adherent culture.

3. The method of claim 1, wherein the population containing pancreatic stem/progenitor cells, is obtained from any tissue which gives rise to pancreatic tissue.

4. The method of claim 1, wherein the population containing pancreatic stem/progenitor cells is obtained from primary gastrointestinal tissue or gastrointestinal derived explant.

5. The method of claim 1, further comprising the steps of further enriching a population from pancreatic or gastrointestinal tissue for pancreatic stem cells, progenitors, or combinations thereof by
c) contacting the selected cells with a second monoclonal antibody that binds CD34 or CD45; and
d) removing those cells that are CD34⁺ or CD45⁺,
wherein the remaining cells in the population are CD34⁻ or CD45⁻ and are enriched for pancreatic stem cells, progenitors, or combinations thereof.

6. The method of claim 1, wherein the pancreatic tissue, pancreatic cells, pancreatic-derived cells, or primary gastrointestinal tissue or gastrointestinal-derived cells are contacted with an anti-CD34 monoclonal antibody or an anti-CD45 monoclonal antibody, and wherein the bound cells are removed prior to the contacting of step a).

7. A method for producing a population enriched for human pancreatic stem cells, progenitors, or a combination thereof, comprising selecting from a population of pancreatic tissue, pancreatic cells, pancreatic-derived cells, or primary gastrointestinal tissue or gastrointestinal-derived cells for those cells that are CD133⁺, or CD49f⁺, or CD133⁺CD49f⁺.

8. The method of claim 7, wherein said selection is accomplished by contacting the population of cells with an anti-CD133 antibody selected from the group consisting of monoclonal antibody AC133 and monoclonal antibody SC111 and removing those cells that do not bind to the anti-CD133 antibody.

9. The method of claim 7, wherein said selection is accomplished by contacting the population of cells with an anti-CD49f antibody selected from the group consisting of monoclonal antibody GoH3 and monoclonal antibody 4F10 and removing those cells that do not bind to the anti-CD49f antibody.

10. The method of claims 8 or 9, further comprising the step of further enriching the population obtained from pancreatic tissue for pancreatic stem cells, progenitors, or combinations thereof by removing the cells that are CD34⁺ from the remaining population of pancreatic tissue, pancreatic cells, pancreatic-derived cells, or primary gastrointestinal tissue or gastrointestinal-derived cells.

11. The method of claim 10, further comprising the step of further enriching the population by removing the cells that bind to a monoclonal antibody that recognizes CD34.

12. The method of claims 8 or 9, further comprising the step of further enriching the population obtained from pancreatic tissue for pancreatic stem cells, progenitors, or combinations thereof by removing the cells that are CD45⁺ from the remaining population of pancreatic tissue, pancreatic cells, pancreatic-derived cells, or primary gastrointestinal tissue or gastrointestinal-derived cells.

13. The method of claim 12, further comprising the step of further enriching the population by removing the cells that bind to a monoclonal antibody that recognizes CD45.

14. A method for enriching from a population of pancreatic tissue, pancreatic cells, pancreatic-derived cells, or primary gastrointestinal tissue or gastrointestinal-derived cells for the populations of pancreatic stem cell or progenitor fractions, comprising selecting from the pancreatic tissue, pancreatic cells, pancreatic-derived cells, or primary gastrointestinal tissue or gastrointestinal-derived cells for cells that express CD133 by binding to monoclonal antibody AC133 or monoclonal antibody SC111, wherein the selected cells are enriched in the fraction of pancreatic stem cells as compared with the population of pancreatic tissue, pancreatic cells, pancreatic-derived cells, or primary gastrointestinal tissue or gastrointestinal-derived cells.

15. A method for enriching a population of pancreatic tissue, pancreatic cells, pancreatic-derived cells, or primary gastrointestinal tissue or gastrointestinal-derived cells for the populations of pancreatic stem cell or progenitor cell fractions, comprising selecting from the pancreatic tissue, pancreatic cells, pancreatic-derived cells, or primary gastrointestinal tissue or gastrointestinal-derived cells for cells that express CD49f by binding to monoclonal antibody GoH3 or monoclonal antibody 4F10, wherein the selected cells are enriched in the fraction of pancreatic stem cells as compared with the population of pancreatic tissue, pancreatic cells, pancreatic-derived cells, or primary gastrointestinal tissue or gastrointestinal-derived cells.

16. The method of claims 14 or 15, further comprising the step of further enriching for the fraction of pancreatic stem cells from primary tissues by removing the cells that are CD34⁺.

17. The method of claims 14 or 15, further comprising the step of further enriching for the fraction of pancreatic stem cells from primary tissues by removing the cells that are CD45⁺.

18. The method of claims 14 or 15, further comprising the step of further enriching for the fraction of pancreatic stem cells from primary tissues by removing the cells that are CD34⁺ and CD45⁺.

19. A method for isolating a pancreatic stem cell from primary pancreatic tissues, comprising:
a) selecting from a population of pancreatic cells, pancreatic-derived cells, or gastrointestinal-derived cells for cells that are CD133⁺, or CD49f⁺, or CD133⁺CD49f⁺;
b) removing the cells that are CD34⁺, CD45⁺, or CD34⁺CD45⁺, wherein the remaining cells are CD34⁻, CD45⁻, or CD34⁻CD45⁻;
c) introducing the cells remaining after step b) to a serum-free culture medium containing one or more growth factors; and
d) proliferating the remaining cells in the culture medium.

20. A method for producing a population enriched for human pancreatic stem cells, progenitors, or combinations thereof comprising selecting from pancreatic tissue, pancreatic cells, pancreatic-derived cells, or primary gastrointestinal tissue or gastrointestinal-derived cells for cells that are CD133⁺ and bind to an anti-CD133 antibody selected from the group consisting of monoclonal antibody AC133 and monoclonal antibody SC111, to produce a population enriched for pancreatic stem cells, progenitors, or combinations thereof, wherein the selecting is by attachment to and disattachment from solid phase.

21. A method for producing a population enriched for human pancreatic stem cells, progenitors, or combinations thereof comprising selecting from pancreatic tissue, pancreatic cells, pancreatic-derived cells, or primary gastrointestinal tissue or gastrointestinal-derived cells for cells that are CD49f⁺ and bind to an anti-CD49f antibody selected from the group consisting of monoclonal antibody GoH3 and monoclonal antibody 4F10, to produce a population enriched for pancreatic stem cells, progenitors, or combinations thereof, wherein the selecting is by attachment to and disattachment from solid phase

22. An antibody that specifically binds to the CD49f antigen, wherein said CD49f antigen specifically binds to the monoclonal antibody GoH3 or to the monoclonal antibody 4F10.

23. The antibody of claim 22, wherein said antibody is a monoclonal antibody produced by a hybridoma cell line.

24. The antibody according to claim 23, wherein said monoclonal antibody binds to the CD49f antigen as detected by the GoH3 antibody or the 4F10 antibody.

25. An antibody that specifically binds to the CD133 antigen, wherein said CD133 antigen specifically binds to the monoclonal antibody AC133 or to the monoclonal antibody SC111.

26. The antibody of claim 25, wherein said antibody is a monoclonal antibody produced by a hybridoma cell line.

27. The antibody according to claim 26, wherein said monoclonal antibody binds to the CD133 antigen as detected by the AC133 antibody or the SC111 antibody.

28. A method for enriching for human pancreatic stem cells, progenitors, or a combination thereof, comprising:
a) combining a population of pancreatic tissue, pancreatic cells, pancreatic-derived cells, or primary gastrointestinal tissue or gastrointestinal-derived cells with a reagent that specifically binds to the CD133 antigen, the CD49f antigen or both the CD133 and CD49f antigens; and
b) selecting for those cells that bind to the reagent,
wherein the selected cells are enriched for pancreatic stem cells, progenitors, or a combination thereof, as compared to the population.

29. The method according to claim 28, wherein said reagent is at least one antibody.

30. A method for producing a population enriched for human pancreatic stem cells, progenitors, or a combination thereof, comprising selecting from pancreatic tissue, pancreatic cells, pancreatic-derived cells, or primary gastrointestinal tissue or gastrointestinal-derived cells for those cells that express CD133 and bind to monoclonal antibody AC133 or to monoclonal antibody SC111, to produce a population enriched for pancreatic stem cells, progenitors, or a combination thereof.

31. A method for producing a population enriched for human pancreatic stem cells, progenitors, or a combination thereof, comprising selecting from pancreatic tissue, pancreatic cells, pancreatic-derived cells, or primary gastrointestinal tissue or gastrointestinal-derived cells for those cells that express CD49f and bind to monoclonal antibody GoH3 or to monoclonal antibody 4F10, to produce a population enriched for pancreatic stem cells, progenitors, or a combination thereof.

32. The method of claim 31, wherein the antibody is monoclonal antibody GoH3.

33. The method of claim 31, wherein the antibody is monoclonal antibody 4F10.

34. The method of claim 30, wherein the antibody is monoclonal antibody AC133.

35. The method of claim 30, wherein the antibody is monoclonal antibody SC111.

36. The method of claim 1, wherein the population containing pancreatic tissue, pancreatic cells, pancreatic-derived cells, or primary gastrointestinal tissue or gastrointestinal-derived cells is obtained from a suspension culture, an adherent monolayer culture, a pancreatic explant, or a gastrointestinal explant.

37. The method of claim 1, wherein the population containing pancreatic tissue, pancreatic cells, pancreatic-derived cells, or primary gastrointestinal tissue or gastrointestinal-derived cells is obtained from primary pancreatic tissue or from primary gastrointestinal tissue.

38. A method for producing a population enriched for human pancreatic stem cells, progenitors, or a combination thereof, wherein the population is obtained from primary pancreas tissues, the method comprising selecting from a population of pancreatic tissue, pancreatic cells, pancreatic-derived cells, or primary gastrointestinal tissue or gastrointestinal-derived cells for cells that are CD133⁺, CD49⁺, or CD133⁺CD49f⁺ the method further comprising the steps of further enriching for pancreatic stem cells, progenitors, or a combination thereof, by further selecting for those cells that are CD34⁻, CD45⁻, or CD34⁻CD45⁻.

39. A method for isolating a pancreatic stem cell, comprising:
a) selecting from a population of primary pancreas tissues, the method comprising selecting from a population of pancreatic tissue, pancreatic cells, pancreatic-derived cells, or primary gastrointestinal tissue or gastrointestinal-derived cells for at least one selected cell that binds to a monoclonal antibody selected from the group consisting of: AC133, SC111, GoH3, and 4F10;
b) introducing at least one selected cell to a serum free culture medium containing one or more growth factors; and
c) proliferating the at least one selected cell in the culture medium.

40. The method of claim 39, wherein the population containing primary pancreas tissues, the method comprising selecting from a population of pancreatic tissue, pancreatic cells, pancreatic-derived cells, or primary gastrointestinal tissue or gastrointestinal-derived cells is obtained from a suspension culture or an adherent culture.

41. The method of claim 39, wherein primary pancreas tissues, the method comprising selecting from a population of pancreatic tissue, pancreatic cells, pancreatic-derived cells, or primary gastrointestinal tissue or gastrointestinal-derived cells is obtained from human fetal, neonatal, juvenile, adult, or postmortem pancreas tissue.

42. The method of claim 39, further comprising the steps of further enriching a population for pancreatic stem cells, progenitors, or a combination thereof, by
d) contacting the selected cells with a second monoclonal antibody that binds to CD34 antigen or CD45 antigen; and
e) removing those cells that are CD34⁺, CD45⁺, or CD34⁺CD45⁺ from the population,
wherein the remaining cells in the population are enriched for pancreatic stem cells, progenitors, or a combination thereof.

43. The method of claim 39, wherein the primary pancreas tissues, the method comprising selecting from a population of pancreatic tissue, pancreatic cells, pancreatic-derived cells, or primary gastrointestinal tissue or gastrointestinal-derived cells are contacted with a monoclonal antibody that binds to CD34 antigen or a monoclonal antibody that binds to CD45 antigen, and wherein the bound cells are removed prior to the contacting of step a).

44. A method of producing a population from pancreatic tissue enriched for human pancreatic lineage committed progenitor and mature cells, comprising:
a) contacting pancreatic cells, pancreatic-derived cells, or gastrointestinal-derived cells with a monoclonal antibody that binds CD9; and
b) selecting said primary pancreas tissues, the method comprising selecting from a population of pancreatic tissue, pancreatic cells, pancreatic-derived cells, or primary gastrointestinal tissue or gastrointestinal-derived cells that bind to the monoclonal antibody;
wherein the selected CD9⁺ cells are enriched for human pancreatic lineage committed progenitor or mature cells capable of differentiating into insulin⁺ β cells.

45. The method of claim 44, further comprising the steps of further enriching a population from pancreatic tissue for human pancreatic lineage committed progenitor cells by
c) contacting the selected cells with a second monoclonal antibody that binds CD15; and
d) removing those cells that are CD15⁺,
wherein the remaining cells in the population are CD15⁻ and are enriched for human pancreatic lineage committed progenitor cells capable of differentiating into insulin⁺β cells.

46. The method of claim 44, wherein the primary pancreas tissues, the method comprising selecting from a population of pancreatic tissue, pancreatic cells, pancreatic-derived cells, or primary gastrointestinal tissue or gastrointestinal-derived cells are contacted with an anti-CD15 monoclonal antibody, and wherein the bound cells are removed prior to the contacting of step a).

47. A method for producing a population enriched for human pancreatic lineage committed progenitor cells, comprising selecting from a population of primary pancreas tissues, the method comprising selecting from a population of pancreatic tissue, pancreatic cells, pancreatic-derived cells, or primary gastrointestinal tissue or gastrointestinal-derived cells for those cells that are CD49f⁺⁺CD9⁺ or CD133⁺CD9⁺.

48. A method for isolating a pancreatic stem cell from primary pancreatic tissues, comprising:
a) selecting from a population of pancreatic cells, pancreatic-derived cells, or gastrointestinal-derived cells for cells that are CD133⁺, CD49f⁺, or CD133⁺CD49f⁺;
b) removing the cells that are CD15+wherein the remaining cells are CD15⁻;
c) introducing the cells remaining after step b) to a serum-free culture medium containing one or more growth factors; and
d) proliferating the remaining cells in the culture medium.

49. A method for producing a population enriched for human pancreatic stem cells, wherein the population is obtained from primary tissues, the method comprising selecting from a population of pancreatic tissue, pancreatic cells, pancreatic-derived cells, or primary gastrointestinal tissue or gastrointestinal-derived cells that are CD133⁺, CD49f⁺, or CD133⁺CD49f⁺ the method further comprising the steps of further enriching for pancreatic stem cells, by further selecting for those cells that are CD15⁻.

50. A pancreatic stem cell, wherein the pancreatic stem cell is CD133⁺, CD49⁺, or

51. A pancreatic progenitor cell committed to the endocrine β-cell lineage, wherein the progenitor cell is CD49f⁺CD9⁺.

52. The endocrine β-cell lineage committed pancreatic progenitor cell of claim 51, wherein the progenitor cell is also CD15⁻.

53. A pancreatic progenitor cell committed to the endocrine β-cell lineage, wherein the progenitor cell is CD49f⁺.

54. A monoclonal antibody which recognizes the CD133 antigen, wherein said monoclonal antibody was deposited at the American Type Tissue Collection on ______, and give the ATCC designation ______.

55. The monoclonal antibody of claim 54, wherein the monoclonal antibody is monoclonal antibody SC111.
